(19) United States
(12) Patent Application Publication
(10) Pub. No.: US 2023/0304102 A1
(43) Pub. Date: Sep. 28, 2023

(54) BIOMARKERS FOR PREDICTING RESPONSIVENESS TO SHP2 INHIBITOR THERAPY

(71) Applicant: D2G Oncology, Inc., Mountain View, CA (US)

(72) Inventors: Ian Paul Winters, Palo Alto, CA (US); Joseph Brian Miranda Juan, Santa Clara, CA (US); Michael Jeremy Rosen, Palo Alto, CA (US); Monte Meier Winslow, Stanford, CA (US); Dmitri Alexandrovich Petrov, San Francisco, CA (US); Gregory Daniel Wall, Burlingame, CA (US)

(21) Appl. No.: 18/161,664

(22) Filed: Jan. 30, 2023

Related U.S. Application Data
(60) Provisional application No. 63/304,875, filed on Jan. 31, 2022.

(51) Int. Cl. C12Q 1/6886

(52) U.S. Cl. CPC C12Q 1/6886; C12Q 2600/106; C12Q 2600/156; C12Q 2600/158

(57) ABSTRACT

The present disclosure provides a method for determining a genotype of one or more biomarker genes in a tumor sample of a subject afflicted with cancer, the method comprising determining a genotype of one or more biomarker genes in the tumor sample and classifying a subject as sensitive or resistant to a therapy comprising a SHP2 inhibitor based on the genotype the one or more biomarker genes in the tumor sample.

### CROSS-REFERENCE TO OTHER APPLICATIONS

This application claims the benefit of US Provisional Application No. 63/304,875, filed Jan. 31, 2022, the full disclosure of which is hereby incorporated by reference herein in its entirety.

### FIELD

Provided herein, in certain aspects, are compositions and methods comprising biomarker genes for identifying subjects that are likely to respond to SHP2 inhibitor therapies.

### STATEMENT OF GOVERNMENTAL INTEREST

This invention was made with government support under Grant 5 R44 CA250672-02 awarded by the National Institutes of Health. The government has certain rights in the invention.

### BACKGROUND

Well balanced levels of tyrosine phosphorylation, maintained by the reversible and coordinated actions of protein tyrosine kinases (PTKs) and protein tyrosine phosphatases (PTPs), are critical for a wide range of cellular processes including growth, differentiation, metabolism, migration, and survival. Aberrant tyrosine phosphorylation as a result of a perturbed balance between the activities of PTKs and PTPs is linked to the pathogenesis of numerous human diseases, including cancer, suggesting that PTPs may be innovative molecular targets for cancer treatment.

PTP family member PTPN11 encodes Src homology region 2 (SH2)-containing protein tyrosine phosphatase-2 (SHP2), an oncogenic tyrosine phosphatase involved in downstream signaling of several receptor tyrosine kinases (RTKs). SHP2 exists in an inactivated form due to an intramolecular allosteric interaction between the N-terminal SH2 domain and the C-terminal phosphatase domain. Phosphorylation at Y542 releases this inhibition and the molecule unfolds, becoming enzymatically active. The phosphatase activity of SHP2 is clearly required for critical cancer-associated signaling pathways such as RAS and phosphatidylinositol 3-kinase (PI3K). SHP2 was traditionally considered “undruggable” because enzymatic active-site inhibitors generally showed off-target inhibition of other proteins and low membrane permeability. More recently, allosteric SHP2 inhibitors with striking inhibitory potency have been developed. These small molecules effectively block the signal transduction between RTKs and RAS/ mitogen-activated protein kinase (MAPK) signaling and have shown efficacy both in preclinical cancer models and subsequent clinical evaluation. Furthermore, the combination of allosteric inhibitors and other kinases inhibitors has been shown to synergistically reduce the possibility of drug resistance. Identifying predictive biomarkers is essential for the future clinical development of SHP2 inhibitors. The present disclosure addresses this need and provides related advantages.

### SUMMARY

Compositions and methods are provided for identifying subjects that are likely to respond to SHP2 inhibitor therapies.

In certain embodiments, the present disclosure provides a method for determining a genotype of one or more biomarker genes comprising EP300, FBXW7, KMT2D, NF1, RB1, RNF43, SETD2, and SMAD4 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the present disclosure provides a method for determining a genotype of one or more biomarker genes comprising EP300, FBXW7, and RB1 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the present disclosure provides a method for determining a genotype of one or more biomarker genes comprising ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the present disclosure provides a method for determining a genotype of one or more biomarker genes comprising ARID1A, BAP1, CREBBP, DICER1, KDM6A, KEAP1, KRASG12D, PALB2, and SMARCA4 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the present disclosure provides a method for determining a genotype of one or more biomarker genes comprising BAP1, CUL3, DICER1, KDM6A, KRAS, NCOA6, NF2, PTEN, SMARCA4, TP53, TSC1, and TSC2 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the present disclosure provides a method for determining a genotype of one or more biomarker genes comprising ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the present disclosure provides a method for determining a genotype of one or more biomarker genes comprising CREBBP, PBRM1, and SMG1 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

In certain embodiments, the methods further comprise (c) classifying the subject as sensitive or resistant to a therapy comprising a SHP2 inhibitor based on the genotype of the one or more biomarker genes in the tumor sample of said subject.

In certain embodiments, the present disclosure provides a method of predicting resistance of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of ARID1A, BAP1, CREBBP, DICER1, KDM6A, KEAP1, KRASG12D, PALB2, and SMARCA4; (ii) a decreased copy number of one or more of ARID1A, BAP1, CREBBP, DICER1, KDM6A, KEAP1, KRASG12D, PALB2, or SMARCA4, or (iii) a decreased expression of mRNA or protein in or more of ARID1A, BAP1, CREBBP, DICER1, KDM6A, KEAP1, KRASG12D, PALB2, and SMARCA4.

A method of predicting resistance of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4; (ii) a decreased copy number of one or more of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4, or (iii) a decreased expression of mRNA or protein in or more of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4.

In certain embodiments, the present disclosure provides a method of predicting resistance of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of BAP1, CUL3, DICER1, KDM6A, KRAS, NCOA6, NF2, PTEN, SMARCA4, TP53, TSC1, and TSC2; (ii) a decreased copy number of one or more of BAP1, CUL3, DICER1, KDM6A, KRAS, NCOA6, NF2, PTEN, SMARCA4, TP53, TSC1, and TSC2, or (iii) a decreased expression of mRNA or protein in or more of BAP1, CUL3, DICER1, KDM6A, KRAS, NCOA6, NF2, PTEN, SMARCA4, TP53, TSC1, and TSC2.

In certain embodiments, the present disclosure provides a method of predicting resistance of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of CREBBP, PBRM1, and SMG1, (ii) a decreased copy number of one or more of CREBBP, PBRM1, and SMG1, or (iii) a decreased expression of mRNA or protein in or more of CREBBP, PBRM1, and SMG.

In certain embodiments, the present disclosure provides a method of predicting resistance of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in PBRM1; (ii) a decreased copy number of PBRM1, or (iii) a decreased expression of mRNA or protein of PBRM1.

In some embodiments, the present disclosure provides a method of enriching a prospective patient population for subjects likely to respond to a SHP2 inhibitor therapy, comprising performing the methods of predicting resistance of tumor growth to inhibition by a therapy comprising a SHP2 inhibitor, as described herein, on two or more individual subjects within the prospective patient population.

In some embodiments, the methods provide a further step of treating cancer in the subject based on the predicted resistance of tumor growth to inhibition by a therapy comprising a SHP2 inhibitor.

In certain embodiments, the present disclosure provides a method of predicting sensitivity of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in one or more of EP300, FBXW7, KMT2D, NF1, RB1, RNF43, SETD2, and SMAD4; (ii) a decreased copy number of one or more of EP300, FBXW7, KMT2D, NF1, RBI, RNF43, SETD2, and SMAD4, or (iii) decreased expression of mRNA or protein in one or more of EP300, FBXW7, KMT2D, NF1, RB1, RNF43, SETD2, and SMAD4.

A method of predicting sensitivity of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in one or more of EP300, FBXW7, and RB1, (ii) a decreased copy number of one or more of EP300, FBXW7, and RBI, or (iii) decreased expression of mRNA or protein in one or more of EP300, FBXW7, and RB1.

A method of predicting sensitivity of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in one or more of ATRX, CDKN2A, DLC1, EP300, FBXW7, KMT2D, NF1, PBRM1, PTPRD, RASA1, RBI, RBM10, RNF43, SETD2, SMAD4, STK11, TET2, and TGFBR2; (ii) a decreased copy number of one or more of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2, or (iii) decreased expression of mRNA or protein in one or more of ATRX, CDKN2A, DLC1, EP300, FBXW7, KMT2D, NF1, PBRM1, PTPRD, RASA1, RBI, RBM10, RNF43, SETD2, SMAD4, STK11, TET2, and TGFBR2.

A method of predicting sensitivity of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in KEAP1, (ii) a decreased copy number of KEAP1, or (iii) decreased expression of mRNA or protein of REAP1.

In some embodiments, the present disclosure provides a method of enriching a prospective patient population for subjects likely to respond to a SHP2 inhibitor therapy, comprising performing the methods of predicting sensitivity of tumor growth to inhibition by a therapy comprising a SHP2 inhibitor, as described herein, on two or more individual subjects within the prospective patient population.

In some embodiments, the methods provide a further step of treating cancer in the subject based on the predicted sensitivity of tumor growth to inhibition by a therapy comprising a SHP2 inhibitor. In some embodiments, the methods comprise treating the subject with a therapy comprising a SHP2 inhibitor.

A method of predicting response of tumor growth to inhibition by a therapy comprising a SHP2 inhibitor, comprising: (a) determining in a tumor sample of a subject a genotype of one or more biomarker genes; (b) analyzing the genotype of the one or more biomarker genes in the tumor sample, and (c) predicting the response of tumor cell growth to inhibition by the therapy comprising a SHP2 inhibitor, if the tumor sample comprises (i) an inactivating mutation in one or more of ARID1A, BAP1, CREBBP, DICER1, KDM6A, KEAP1, KRASG12D, PALB2, and SMARCA4; (ii) a decreased copy number of one or more of ARID1A, BAP1, CREBBP, DICER1, KDM6A, KEAP1, KRASG12D, PALB2, and SMARCA4; or (iii) a decreased expression of mRNA or protein in or more of ARID1A, BAP1, CREBBP, DICER1, KDM6A, KEAP1, KRASG12D, PALB2, and SMARCA4; (iv) an inactivating mutation in one or more of EP300, FBXW7, KMT2D, NF1, RBI, RNF43, SETD2, and SMAD4; (v) a decreased copy number of one or more of EP300, FBXW7, KMT2D, NF1, RBI, RNF43, SETD2, and SMAD4, or (vi) a decreased expression of mRNA or protein in one or more of EP300, FBXW7, KMT2D, NF1, RBI, RNF43, SETD2, and SMAD4.

A method of predicting response of tumor growth to inhibition by a therapy comprising a SHP2 inhibitor, comprising: (a) determining in a tumor sample of a subject a genotype of one or more biomarker genes; (b) analyzing the genotype of the one or more biomarker genes in the tumor sample, and (c) predicting the response of tumor cell growth to inhibition by the therapy comprising a SHP2 inhibitor, if the tumor sample comprises (i) an inactivating mutation in one or more of BAP1, CML3, DICER1, KDM6A, KRAS, NCOA6, NF2, PTEN, SMARCA4, TP53, TSC1, and TSC2; (ii) a decreased copy number of one or more of BAP1, CUL3, DICER1, KDM6A, KRAS, NCOA6, NF2, PTEN, SMARCA4, TP53, TSC1, and TSC2; or (iii) a decreased expression of mRNA or protein in or more of BAP1, CUL3, DICER1, KDM6A, KRAS, NCOA6, NF2, PTEN, SMARCA4, TP53, TSC1, and TSC2; (iv) an inactivating mutation in one or more of EP300, FBXW7, and RB1, (v) a decreased copy number of one or more of EP300, FBXW7, and RBI, or (vi) a decreased expression of mRNA or protein in one or more of EP300, FBXW7, and RB1.

A method of predicting response of tumor growth to inhibition by a therapy comprising a SHP2 inhibitor, comprising: (a) determining in a tumor sample of a subject a genotype of one or more biomarker genes; (b) analyzing the genotype of the one or more biomarker genes in the tumor sample, and (c) predicting the response of tumor cell growth to inhibition by the therapy comprising a SHP2 inhibitor, if the tumor sample comprises (i) an inactivating mutation in one or more CREBBP, PBRM1, and SWG1; (ii) a decreased copy number of one or more of CREBBP, PBRM1, and SMG1, or (iii) a decreased expression of mRNA or protein in or more of CREBBP, PBRM1, and SMG1, (iv) an inactivating mutation in KEAP1, (v) a decreased copy number of KE4P1, or (vi) a decreased expression of mRNA or protein in KEAP1.

In some embodiments, the present disclosure provides a method of enriching a prospective patient population for subjects likely to respond to a SHP2 inhibitor therapy, comprising performing the methods of predicting response of tumor growth to inhibition by a therapy comprising a SHP2 inhibitor, as described herein, on two or more individual subjects within the prospective patient population.

In some embodiments, the methods provide a further step of treating cancer in the subject based on the predicted response of tumor growth to inhibition by a therapy comprising a SHP2 inhibitor. In some embodiments, the methods comprise treating the subject with a therapy comprising a SHP2 inhibitor. In some embodiments, the methods comprise treating the subject with a therapy comprising a therapeutic agent other than a SHP2 inhibitor.

In some embodiments, the present disclosure provides a method of treating cancer in a subject, comprising treating a subject with a SHP2 inhibitor when a tumor sample of the subject comprises (i) an inactivating mutation in one or more of CDKN2A, EP300, KRAS, MGA, EP300, FBXW7, or RB1, (ii) a decreased copy number of one or more of EP300, FBXW7, or RBI, or (iii) decreased expression of mRNA or protein in one or more of EP300, FBXW7, or RB1. In some embodiments, the tumor sample of the subject further comprises absence of (i) an inactivating mutation in one or more of BAP1, CUL3, DICER1, KDM6A, KRAS, NCOA6, NF2, PTEN, SMARCA4, TP53, TSC1, or TSC2, (ii) a decreased copy number of one or more of BAP1, CUL3, DICER1, KDM6A, KRAS, NCOA6, NF2, PTEN, SMARCA4, TP53, TSC1, or TSC2, or (iii) decreased expression of BAP1, CUL3, DICER1, KDM6A, KRAS, NCOA6, NF2, PTEN, SMARCA4, TP53, TSC1, or TSC2 mRNA or protein.

In some embodiments, the present disclosure provides a method of treating cancer in a subject, comprising treating a subject with a SHP2 inhibitor when a tumor sample of the subject comprises (i) an inactivating KEAP1 mutation, (ii) a decreased copy number of KEAP1, or (iii) decreased expression of KEAP1 mRNA or protein. In some embodiments, the tumor sample of the subject further comprises absence of (i) an inactivating mutation in one or more of CREBBP, PBRM1, or SMG1, (ii) a decreased copy number of one or more of CREBBP, PBRM1, or SMG1, or (iii) a decreased expression of CREBBP, PBRM1, or SMG1 mRNA or protein. In some embodiments, the tumor sample of the subject further comprises absence of (i) an inactivating mutation in PBRM1, (ii) a decreased copy number of PBRM1, or (iii) a decreased expression of PBRM1 mRNA or protein.

In some embodiments, the present disclosure provides a composition comprising one or more isolated biomarker genes selected from the group consisting of ARID1A, BAP1, CREBBP, DICER1, KDM6A, KEAP1, KRASG12D, PALB2, and SMARCA4. In some embodiments, the present disclosure provides a composition comprising one or more isolated biomarker genes selected from the group consisting of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4. In some embodiments, the present disclosure provides a composition comprising one or more isolated biomarker genes selected from the group consisting of CREBBP, PBRM1, and SMG1. In some embodiments, the present disclosure provides a composition comprising one or more isolated biomarker genes selected from the group consisting of EP300, FBXW7, and RB1. In some embodiments, the present disclosure provides a composition comprising one or more isolated biomarker genes selected from the group consisting of EP300, FBXW7, KMT2D, NF1, RBI, RNF43, SETD2, and SMAD4. In some embodiments, the present disclosure provides a composition comprising one or more isolated biomarker genes selected from the group consisting of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2.

In some embodiments, the present disclosure provides a method of detecting one or more isolated biomarker genes selected from the group consisting of ARID1A, BAP1, CREBBP, DICER1, KDM6A, KEAP1, KRASG12D, PALB2, and SMARCA4 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the methods further comprise obtaining a tumor sample from the subject.

In some embodiments, the present disclosure provides a method of detecting one or more isolated biomarker genes selected from the group consisting of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4 in a a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the methods further comprise obtaining a tumor sample from the subject.

In some embodiments, the present disclosure provides a method of detecting one or more isolated biomarker genes selected from the group consisting of BAP1, CUL3, DICER1, KDM6A, KRAS, NCOA6, NF2, PTEN, SMARCA4, TP53, TSC1, and TSC2 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the methods further comprise obtaining a tumor sample from the subject.

In some embodiments, the present disclosure provides a method of detecting one or more isolated biomarker genes selected from the group consisting of CREBBP, PBRM1, and SMG1 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the methods further comprise obtaining a tumor sample from the subject.

In some embodiments, the present disclosure provides a method of detecting one or more isolated biomarker genes selected from the group consisting of EP300, FBXW7, and RB1 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the methods further comprise obtaining a tumor sample from the subject.

In some embodiments, the present disclosure provides a method of detecting one or more isolated biomarker genes selected from the group consisting of EP300, FBXW7, KMT2D, NF1, RBI, RNF43, SETD2, and SMAD4 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the methods further comprise obtaining a tumor sample from the subject.

In some embodiments, the present disclosure provides a method of detecting one or more isolated biomarker genes selected from the group consisting of ATRX, CDKN2A, DLC1, EP300, FBXW7, KMT2D, NF1, PBRM1, PTPRD, RASA1, RBI, RBM10, RNF43, SETD2, SMAD4, STK11, TET2, and TGFBR2 in a subject, comprising detecting whether the one or more isolated biomarker genes are present in the tumor sample by contacting a tumor sample of the subject with a binding agent and detecting binding between the one or more isolated biomarker genes and the binding agent. In certain embodiments, the methods further comprise obtaining a tumor sample from the subject.

In some embodiments, the subject is classified as sensitive or resistant to a therapy comprising a SHP2 inhibitor based on the methods of detecting one or more isolated biomarker genes described herein. Accordingly, in some embodiments, the methods of the present disclosure described herein further comprise classifying a subject as sensitive or resistant to a therapy comprising a SHP2 inhibitor.

In some embodiments, the present disclosure provides a method of determining effectiveness of a SHP2 inhibitor in reducing tumor size comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; (e) comparing sizes of the first and second inert tumors after the therapy; (f) comparing sizes of the first and second mutant tumors after completion of the therapy, and (g) identifying the mutant tumor genotype as sensitive to the SHP2 inhibitor if the change in tumor size between the first and second inert tumors after the therapy is less than the change in tumor size between the first and second mutant tumors after the therapy.

A method of determining effectiveness of a SHP2 inhibitor in reducing tumor size comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; (e) comparing sizes of the first and second inert tumors after the therapy; (f) comparing sizes of the first and second mutant tumors after completion of the therapy, and (g) identifying the mutant tumor genotype as resistant to the SHP2 inhibitor if the change in tumor size between the first and second inert tumors after the therapy is greater than the change in tumor size between the first and second mutant tumors after the therapy.

In some embodiments, the subject is a human subject.

In some embodiments, the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma and cholangiocarcinoma. In some embodiments, the cancer is non-small cell lung cancer (NSCLC).

In some embodiments, the cancer is a KRAS-mutant cancer.

In some embodiments, the tumor sample comprises a mutation in at least one gene. In some embodiments, the gene is a tumor suppressor gene. In some embodiments, the tumor suppressor gene is TP53. In some embodiments, the tumor sample has been previously genotyped and determined to comprise a mutation in at least one gene. In some embodiments, the gene is an oncogene. In some embodiments, the oncogene is KRAS. In some embodiments, the methods of the present disclosure described herein further comprise administering a SHP2 inhibitor therapy to the subject.

In some embodiments, the methods of the present disclosure described herein further comprise administering to the subject a second therapy. In some embodiments, the second therapy comprises an agent that affects KRAS activity between inactive guanosine diphosphate (GDP)-bound and active guanosine triphosphate (GTP)-bound states. In some embodiments, the second therapy comprises an agent that targets the immune system directly.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a biomarker heatmap showing the study of pharmacogenomic interactions (PGx) of SHPi with inactivation of tumor suppressor genes. Relative tumor number (RTN) > 0 indicates drug resistance, and RTN = 1 corresponds to 2× change in tumor number (larger than each cutoff) relative to change in untreated vs. treated for oncogene-only tumors, while RTN = -1 corresponds to 0.5× change and drug sensitivity. *: p<0.05 and +: p<0.2. Both p-values are two-tailed and based on fraction of bootstraps with RTN scores great or less than 0. Missing cells in heatmap correspond to genotypes that were not assayed in the particular study.

FIG. 2 shows a bar graph depicting aggregated RTN scores and their corresponding 95% confidence intervals for SHP2 inhibitor therapy. Shading indicates the respective classification memberships, which was defined as follows. Resistant or Sensitive: Family-wise error rate (FWER) less or equal to 0.05 and absolute value of RTN score greater than 0.1. Extended: false discovery rate (FDR) less than or equal to 0.1 and absolute value of RTN score greater than 0.08.

### DETAILED DESCRIPTION

The present present disclosure is based, at least in part, on the surprising discovery that the genotype of particular biomarker genes can be used to predict a subject’s response to a SHP2 inhibitor therapy. In certain embodiments, the genotype is predictive of sensitivity to a SHP2 inhibitor therapy. In certain embodiments, the genotype is predictive of resistance to a SHP2 inhibitor therapy. The present disclosure herein provides new and advantageous methods for determining whether a subject afflicted with cancer is a candidate for a SHP2 inhibitor therapy.

Before the present methods and compositions are described in greater detail, it is to be understood that the present disclosure is not limited to a particular method or composition described, and as such may, of course, vary. It is also to be understood that the terminology used herein is for the purpose of describing particular embodiments only, and is not intended to be limiting, since the scope of the present present disclosure will be limited only by the claims.

Where a range of values is provided, each intervening value, to the tenth of the unit of the lower limit unless the context clearly dictates otherwise, between the upper and lower limits of that range is also specifically disclosed.

Unless defined otherwise, all technical and scientific terms used herein have the same meaning as commonly understood by one of ordinary skill in the art to which the present disclosure belongs. Although any methods and materials similar or equivalent to those described herein can be used in the practice or testing of the present present disclosure, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

In certain embodiments, the present disclosure provides a method comprising: (a) determining the genotype of one or more biomarker genes in a tumor sample obtained from a subject; (b) identifying whether the subject is likely to be sensitive to a therapy comprising a SHP2 inhibitor based on the genotype. In certain embodiments, the present disclosure provides a method comprising: (a) determining the genotype of one or more biomarker genes in a tumor sample of a subject; (b) identifying whether the subject is likely to be sensitive to a therapy comprising a SHP2 inhibitor based on the genotype. In certain embodiments, the methods further comprise administering to the subject the SHP2 inhibitor if the subject is identified as likely to be sensitive to a therapy comprising a SHP2 inhibitor.

In certain embodiments, the biomarker genes useful in the methods of the present disclosure comprise one or more of APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRAM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In certain embodiments, the biomarker genes useful in the methods of the present disclosure comprise one or more, two or more, three or more, four or more, five or more, six or more, seven or more, eight to more biomarker genes selected from the group consisting of ARID1A, BAP1, CREBBP, DICER1, KDM6A, KEAP1, KRASG12D, PALB2, and SMARCA4.

In certain embodiments, the biomarker genes useful in the methods of the present disclosure comprise one or more, two or more, three or more, four or more, five or more, six or more, seven or more, eight to more, nine or more, ten or more, eleven or more, twelve or more, thirteen or more, fourteen or more, fifteen or more, sixteen or more, seventeen or more, eighteen or more biomarker genes selected from the group consisting of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4.

In certain embodiments, the biomarker genes useful in the methods of the present disclosure comprise one or more, two or more, three or more, four or more, five or more, six or more, seven or more biomarker genes selected from the group consisting of EP300, FBXW7, KMT2D, NF1, RBI, RNF43, SETD2, and SMAD4.

In certain embodiments, the biomarker genes useful in the methods of the present disclosure comprise one or more, two or more, three or more, four or more, five or more, six or more, seven or more, eight to more, nine or more, ten or more, eleven or more, twelve or more, thirteen or more, fourteen or more, fifteen or more, sixteen or more, seventeen or more, eighteen or more, nineteen or more, twenty or more biomarker genes selected from the group consisting of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2.

It must be noted that as used herein and in the appended claims, the singular forms “a,” “an,” and “the” include plural reference unless the context clearly dictates otherwise.

It should also be understood that the terms “about,” “approximately,” “generally,” “substantially,” and like terms, used herein when referring to a dimension or characteristic of a component of embodiments provided herein, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (for example, rounding, measurement or other systematic errors, manufacturing tolerances, etc.), would not vary the least significant digit. Unless otherwise stated, any numerical values, such as a concentration or a concentration range described herein, are to be understood as being modified in all instances by the term “about.”

Unless otherwise indicated, the term “at least” preceding a series of elements is to be understood to refer to every element in the series and any one or any and all combinations of the elements.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure, or composition.

As used herein, the term “subject” refers to any organism, for example, a mammal, for whom diagnosis, prognosis, or therapy is desired. Mammalian subjects include humans, domestic animals, farm animals, sports animals, and zoo animals including, for example, humans, non-human primates, dogs, cats, guinea pigs, rabbits, rats, mice, horses, cattle, and so on.. In certain embodiments, the subject has been diagnosed with cancer.

In some embodiments, the subject is afflicted with cancer and has been diagnosed with a need for treatment for cancer. In some embodiments, the subject has been previously determined to have a driver mutation selected from the group consisting of Kirsten rat sarcoma (KRAS), epidermal growth factor receptor (EGFR), MET, anaplastic lymphoma kinase (ALK), c-ROS oncogene 1 (ROSI), v-raf murine sarcoma viral oncogene homolog B (BRAF), neurotrophic receptor tyrosine kinase (NTRK), human epidermal growth factor 2 (HER2), neuregulin-1 (NRG1) and rearranged during transfection (RET). In some embodiments, the subject has been previously determined to have a KRAS-driven, also referred to as a KRAS-mutated, cancer. In some embodiments, a tumor sample of a subject has been previously genotyped and confirmed the presence of a driver mutation.

In some embodiments, the subject has one or more inactivated tumor suppressor genes. In some embodiments, the subject has been previously determined to have an inactivated p53 gene. The involvement of p53 inactivation at various stages of tumorigenesis and specific contribution of p53 mutations at each phase of cancer progression have been described. (Rivlin et al. Genes & Cancer 2(4):466-74 (2011)). In some embodiments, a tumor sample of a subject has been previously genotyped and confirmed the presence of a mutation in a tumor suppressor gene.

The terms “inhibit,” “block,” and “suppress” are used interchangeably and refer to any statistically significant decrease in a tumor activity, including full blocking of the activity. An “inhibitor” is an active agent that inhibits, blocks, or suppresses tumor activity in vitro or in vivo. Inhibitors include but are not limited to small molecule compounds; nucleic acids, such as siRNA and shRNA; polypeptides, such as antibodies or antigen-binding fragments thereof, dominant-negative polypeptides, and inhibitory peptides; and oligonucleotide or peptide aptamers.

As used herein, the term “SHP2 inhibitor” or “SHP2i” refers to any active agent that antagonizes the activity of a SHP2 protein, reduces its production or activity in a cell. The development of SHP2 inhibitors has been systematically reviewed, for example, by Song et al., Acta Pharm Sin B, 11 (2020), pp. 13-29; Yuan et al., J Med Chem, 63 (2020), pp. 11368-11396; Tang et al., Eur J Med Chem, 204 (2020), p. 112657, and Shen et al., Eur J Med Chem, 190 (2020), p. 112117.

Any suitable SHP2 inhibitor can be used in the methods described herein. Exemplary SHP2 inhibitors include, for example, ERAS-601 (Erasca), BBP-398 (BridgeBio), RMC-4630 (Rev Med/Sanofi), RMC-4550 (Rev Med), JAB-3068 (Jacobio Pharmaceuticals Co., Ltd., AbbVie), and JAB-3312 (Jacobio Pharmaceuticals Co., Ltd., AbbVie), RLY-1971 (Hoffmann-La Roche), PF-07284892 (Pfizer), TNO-155 (Novartis), ET0038 (Etern BioPharma (Shanghai) Co., Ltd), HBI-2376 (HUYABIO International, LLC), HS-10381 (Jiangsu Hansoh Pharmaceutical Co., Ltd.), and SH3809 (Nanjing Sanhome Pharmaceutical, Co., Ltd.).

In certain embodiments, the SHP2 inhibitor is a small molecule. In some embodiments, the SHP2 inhibitor is a polypeptide. In certain embodiments, the SHP2 inhibitor is a polypeptide analog. In certain embodiments, the SHP2 inhibitor is a peptidomimetic. In certain embodiments, the SHP2 inhibitor is an aptamer.

In certain embodiments, a SHP2 inhibitor is an antibody or an antigen-binding fragment thereof. For example, the antibody or antigen-binding fragment thereof can be a humanized antibody, a recombinant antibody, a diabody, a chimerized or chimeric antibody, a monoclonal antibody, a deimmunized antibody, a fully human antibody, a single chain antibody, an Fᵥ fragment, an F_{d} fragment, a Fab fragment, a Fab′ fragment, or an F(ab′)₂ fragment.

Any compound which binds to and inhibits, or otherwise inhibits the activity, function and/or the expression of a SHP2 protein or its receptor can be utilized in accordance with the present disclosure. For example, an inhibitor of a SHP2 protein can be, for example, a small molecule, a nucleic acid or nucleic acid analog, a peptidomimetic, or a macromolecule that is not a nucleic acid or a protein. Accordingly, compounds which can be utilized as SHP2 inhibitors include, but are not limited to, proteins, protein fragments, peptides, small molecules, RNA aptamers, L-RNA aptamers, spiegelmers, antisense compounds, serine protease inhibitors, molecules which can be utilized in RNA interference (RNAi) such as double stranded RNA including small interfering RNA (siRNA), locked nucleic acid (LNA) inhibitors, peptide nucleic acid (PNA) inhibitors, etc.

A SHP2 inhibitor of can also be, for example, a small molecule, a polypeptide analog, a nucleic acid, or a nucleic acid analog.

“Small molecule” as used herein, is meant to refer to an agent, which has a molecular weight of less than about 6 kDa, e.g., less than about 2.5 kDa. Many pharmaceutical companies have extensive libraries of chemical and/or tumor mixtures comprising arrays of small molecules, often fungal, bacterial, or algal extracts, which can be screened with any of the assays of the application. It is within the scope of this application that such a library can be used to screen for agents that bind to a target antigen of interest (for example, a SHP2 protein). There are numerous commercially available compound libraries, such as the Chembridge DIVERSet® screening library. Libraries are also available from academic and governmental entities, such as the National Cancer Institute’s Developmental Therapeutics Program (DTP). Rational drug design can also be employed and can be achieved based on known compounds, for example, a known inhibitor of a SHP2 protein (for example, an antibody, or antigen-binding fragment thereof, that binds to a SHP2 protein).

In certain embodiments, the SHP2 inhibitor is an antibody, or antigen-binding fragment thereof, which binds to a SHP2 protein.

As used herein, the term “antibody” is used in a broad sense and includes immunoglobulin or antibody molecules including human, humanized, composite and chimeric, single-chain, bi-specific and multi-specific antibodies, and antibody fragments, in particular, antigen-binding fragments, that are monoclonal or polyclonal. In general, antibodies are proteins or peptide chains that exhibit binding specificity to a specific antigen. Antibody structures are well known. Immunoglobulins can be assigned to five major classes (specifically, IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding subclasses. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains.

In certain embodiments, the SHP2 inhibitor is a nucleic acid inhibitor. Nucleic acid inhibitors can be used to bind to and inhibit a target antigen of interest. The nucleic acid antagonist can be, for example, an aptamer or a small interfering RNA (siRNA). Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets affinities and specificities similar to those of antibodies.

By introducing a certain nucleic acid modality to the desired tissue of the patient, SHP2 gene expression can be downregulated, augmented, or corrected. Small interfering RNA (siRNA), microRNA (miRNA) and inhibitory antisense oligonucleotides (ASOs) are representative molecules used to trigger gene inhibition, whereas plasmid DNA, messenger RNA (mRNA), small activating RNA (saRNA), splicing-modulatory ASOs and CRISPR (clustered regularly interspaced short palindromic repeats)/Cas (CRISPR-associated protein) systems are usually employed to increase or correct target gene expression.

In certain embodiments, the SHP2 inhibitor is a non-antibody scaffold protein. These proteins are, generally, obtained through combinatorial chemistry-based adaptation of pre-existing antigen-binding proteins. For example, the binding site of human transferrin for human transferrin receptor can be modified using combinatorial chemistry to create a diverse library of transferrin variants, some of which have acquired affinity for different antigens. The portion of human transferrin not involved with binding the receptor remains unchanged and serves as a scaffold, like framework regions of antibodies, to present the variant binding sites. The libraries are then screened, as an antibody library is, against a target antigen of interest to identify those variants having optimal selectivity and affinity for the target antigen. Non-antibody scaffold proteins, while similar in function to antibodies, are touted as having a number of advantages as compared to antibodies, which advantages include, among other things, enhanced solubility and tissue penetration, less costly manufacture, and ease of conjugation to other molecules of interest. Hey et al. (2005) TRENDS Biotechnol 23(10):514-522.

As used herein, the term “inhibit” or “inhibiting” or “reducing” includes the decrease, limitation or blockage of, for example, a particular action, function, or interaction. For example, in the context of tumor growth, “inhibited” means terminated, reduced, delayed, or prevented. Tumor growth is also “inhibited” if recurrence or metastasis of the cancer is reduced, slowed, delayed, or prevented.

As used herein, “reducing the tumor,” means reducing the size, volume, or weight of the tumor, reducing the number of metastases, reducing the size or weight of a metastasis, or combinations thereof. In some examples a metastasis is cutaneous or subcutaneous. Thus, in some embodiments, administration of the SHP2 inhibitor reduces the size and/or volume of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, administration of the SHP2 inhibitor reduces the weight of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, administration of the SHP2 inhibitor reduces the size or volume of a metastasis by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, administration of the SHP2 inhibitor reduces the number of metastases by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, combinations of these effects are achieved.

In certain embodiments, the methods described herein further comprise administering to the subject a second therapy in combination with SHP2 inhibition. In some apects, the second therapy targets KRAS activity between inactive guanosine diphosphate (GDP)-bound and active guanosine triphosphate (GTP)-bound states. In some emdiments, the second therapy targets the Kirsten rat sarcoma viral (KRAS) oncogene, for example, with a covalent KRASG12C inhibitor such as sotorasib or adagrasib. KRAS inhibitors are well known in the art and have been extensively reviewed, for example, by Wang et al., European Journal of Medicinal Chemistry (2023) 249: 115124. In some embodiments, the second therapy targets Son of Sevenless (SOS), a guanine nucleotide exchange factor that catalyzes the switch from the KRAS(off) to the KRAS(on) conformation. In some embodiments, the second therapy targets growth factor receptor-bound protein 2 (GRB2).

In some embodiments, the second therapy comprises a kinase inhibitor. As of 2023, there are about 72 FDA-approved drugs that target about two dozen different protein kinases as described, for example, by Roskoski (2023). Pharmacological Research 187:106552. Kinase inhibitors, including their role in cancer therapy, have been comprehensively described and reviewed in the art. (Cohen et al. (2021) Nature Reviews Drug Discovery 20: 551-569). A kinase inhibitor suitable for combination with a SHP2 inhibitor in the methods described herein can target, for example, a protein-serine/threonine protein kinase, a dual specificity protein kinase (MEK1/2), a nonreceptor protein-tyrosine kinase, or a target receptor protein-tyrosine kinase. In some embodiments, the second therapy comprises immunotherapy.

In some embodiments, the combination therapy is directed to overcoming drug resistance using a SHP2 inhibitor and the second therapy that is selected from immunochemotherapy, dual target inhibitors, dual allosteric inhibitors, covalent inhibitors, and the combined use of allosteric inhibitors and orthosteric inhibitors, proteolysis targeting chimeras (PROTACs) technology to degrade mutant proteins. In certain embodiments, the second therapy further comprises an agent that targets the immune system directly, such as a PD-1 inhibitor and a colony stimulating factor 1 receptor (CSF-1R) inhibitor.

In certain embodiments, the agent is an immune checkpoint inhibitor. In certain embodiments, the immune checkpoint inhibitor inhibits programmed cell death protein 1 (PD-1). In certain embodiments, the immune checkpoint inhibitor is an anti-PD-1 antibody. In certain embodiments, the immune checkpoint inhibitor selected from the group consisting of nivolumab (Opdivo), Pembrolizumab (Keytruda), and Cemiplimab (Libtayo). In certain embodiments, the immune checkpoint inhibitor inhibits cytotoxic T-lymphocyte-associated antigen 4 (CTLA-4). In certain embodiments, the immune checkpoint inhibitor is an anti-CTLA-4 antibody. In certain embodiments, the immune checkpoint inhibitor is Ipilimumab (Yervoy). In certain embodiments, the immune checkpoint inhibitor inhibits programmed cell death protein 1 ligand (PD-L1). In certain embodiments, the immune checkpoint inhibitor is an anti-PD-L1 antibody. In certain embodiments, the immune checkpoint inhibitor is selected from the group consisting of Atezolizumab (Tecentriq), avelumab (Bevencio), durvalumab (Imfinzi).

A “biomarker gene” can be any gene having a genotype and/or expression level that can be determined, measured and/or evaluated as an indicator of a biologic process, pathogenic process, or pharmacologic response to a therapeutic intervention. A biomarker gene useful to practice the presently disclosed methods can be used as an indicator to determine whether a subject having a tumor (e.g., a cancer) will be sensitive or resistant to a therapy comprising a SHP2 inhibitor and/or for monitoring response to a treatment with a therapy comprising a SHP2 protein inhibitor. In certain embodiments, a biomarker gene is a tumor suppressor gene. Sensitivity or resistance to a therapy with a SHP2 protein inhibitor can be determined by analyzing a nucleic acid molecule (DNA, mRNA, cDNA etc.) corresponding to a biomarker gene or the protein encoded by the biomarker gene. Biomarker genes can include any gene whose genotype and/or level of expression in a tissue or cell can be used to predict response to a SHP2 inhibitor therapy. The detection, and in some cases the level, of one or more biomarker genes of the present disclosure permit the classification of a subject as sensitive or resistant to a SHP2 inhibitor therapy.

In some embodiments, a biomarker gene is a tumor suppressor gene. In some embodiments, a biomarker gene is part of a pathway or otherwise related to one of the biomarker genes disclosed herein. Additional biomarker genes useful in the methods disclosed herein can be identified by those skilled in the art based on the present disclosure. See, for example, Itatani et al., Int J Mol Sci., 20(23):5822, (2019)(TGF-β signaling pathway); Manning et al., Genes Dev., 19(15):1773-1778, (2005) (PI3K-Akt pathway); Johannessen et al., Proc Natl Acad Sci, 102(24): 8573-8578, (2005) (PI3K-Akt pathway); Tsukiyama et al., Mol Cell Biol, 35:2007-2023, (2015)(Wnt signaling pathway); Zhang et al., Molecular Cancer, 17, 45 (2018)(c-Met signaling pathway); Mombach et al., BMC Genomics., 15 Suppl 7(Suppl 7):S7 (2014) (G1/S checkpoint); Shain et al., PLoS ONE, 8(1): e55119, (2013)(SWI/SNF complex); Iyer et al., Oncogene, 23:4225-4231, (2004) (p300 and CBP); Villeneuve etal., Mol Cell., 51(1): 68-79, (2013)(USP15, KEAP1, and CUL3); Sahtoe et al., Nat Commun 7, 10292 (2016) (BAP1 and ASXL1); Harris et al., Oncogene, 24:2899-2908 (2005)(p53).

As used herein, the terms “determine,” “determine the genotype of a biomarker gene,” “determine the level of a biomarker gene,” “determine the amount of a biomarker gene,” “determine the biomarker gene level,” and the like are meant to encompass any technique that can be used to detect or measure the genotype, presence, or expression level of one or more biomarker genes. Such techniques can give qualitative or quantitative results. Biomarker gene levels can be determined by detecting the entire biomarker molecule or by detecting fragments or reaction products that are characteristic of the biomarker gene. The terms determining, measuring or taking a measurement refer to a quantitative or qualitative determination of a property of an entity, for example, quantifying the amount or concentration of a molecule or the activity level of a molecule. Any known method of detecting or measuring the level of a biomarker can be used to practice the present disclosure, so long as the method detects the genotype, presence, absence, or expression level of the biomarker gene.

In certain embodiments, determining the genotype of a biomarker gene is performed at the nucleic acid level by performing RNA-seq, a reverse transcriptase polymerase chain reaction (RT-PCR) or a hybridization assay with oligonucleotides that are substantially complementary to portions of cDNA molecules of the at least one biomarker gene under conditions suitable for RNA-seq, RT-PCR or hybridization and obtaining expression levels of the at least one biomarker gene.

As used herein, the terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist isolated within an animal, or can be non-tumorigenic, such as a leukemia cell. Cancers include, but are not limited to, B cell malignancies, for example, multiple myeloma,, the heavy chain diseases, such as, for example, alpha chain disease, gamma chain disease, and mu chain disease, benign monoclonal gammopathy, and immunocytic amyloidosis, skin cancer, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms’ tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, for example, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin’s disease and non-Hodgkin’s disease), multiple myeloma, Waldenstrom’s macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (for example, serous ovarian carcinoma), or breast carcinoma. The amount of a tumor in an individual is the “tumor burden” which can be measured as the number, volume, and/or weight of the tumor.

Non-limiting exemplary cancers in the embodiments of the present disclosure include skin cancer, lung cancer, pancreatic cancer, breast cancer, colorectal cancer, bladder cancer, liver cancer, kidney cancer, leukemia, and lymphoma. In some embodiments, the cancer is an advanced solid tumor. In some embodiments, the cancer is selected from the group consisting of lung cancer, pancreatic cancer, colorectal cancer, ovarian cancer, urothelial carcinoma, B cell lymphoma, chronic lymphocytic leukemia (CLL), head and neck squamous cell carcinoma (HNSCC), metastatic castration-resistant prostate cancer (mCRPC), and prolymphocytic leukemia (PLL). In some embodiments, the cancer is lung cancer. In some embodiments, the lung cancer is non-small cell lung cancer (NSCLC). In some embodiments, NSCLC is lung adenocarcinoma. In some embodiments, the cancer is pancreatic cancer. In some embodiments, the cancer is colorectal cancer.

As used herein, the term “lung cancer” refers to the collection of cancers affecting lung tissue. Non-small cell lung cancer (NSCLC) represents approximately 87% of all lung cancers, with the remaining 13% of cases being small-cell lung cancer (SCLC). There are three main types of NSCLC: squamous cell carcinoma (approximately 30 percent)., large cell carcinoma (approximately 40 percent)., and adenocarcinoma (approximately 15 percent).

The term “classifying” includes associating a sample with a response to a SHP2 inhibitor therapy. In certain instances, “classifying” is based on statistical evidence, empirical evidence, or both. In certain embodiments, the methods of classifying utilize a training set of samples having known genotypes. Once established, the training data set can serve as a basis, model, or template against which the features of an unknown sample are compared, in order to classify the sample.

The term “control” refers to any reference standard suitable to provide a comparison to the expression products in the test sample. A control can comprise a reference standard expression product level or genotype score from any suitable source, including but not limited to housekeeping genes, an expression product level range from normal tissue (or other previously analyzed control sample), a previously determined expression product level range within a test sample of a group of patients, or a set of patients with a certain outcome or receiving a certain therapy. It will be understood by those of ordinary skill in the art that such control samples and reference standard expression product levels can be used in combination as controls in the methods of the present disclosure. In various embodiments, the biomarker gene expression can be compared to a reference. A “reference” can be any value derived by art known methods for establishing a reference.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed protein can be within the cytoplasm of a host cell, into the extracellular milieu such as the growth medium of a cell culture or anchored to the cell membrane.

The term “gene product” as used herein, refers to RNA transcribed from a gene and to one or more proteins, polypeptides of fragments thereof that are the product of translation of the RNA transcribed from the gene, and further encompasses all naturally occurring post-transcriptional and post-translational modifications. The expressed protein can be within the cytoplasm of a host cell, into the extracellular milieu such as the growth medium of a cell culture or anchored to the cell membrane.

The terms “expression level” and “level of expression” as used herein refers to information regarding the relative or absolute level of expression of one or more biomarker genes in a cell or group of cells. The level of expression of a biomarker gene can be determined based on the level of RNA, such as mRNA, encoded by the gene. Alternatively, the level of expression can be determined based on the level of a polypeptide or fragment thereof encoded by the biomarker gene. Gene expression data can be acquired for an individual cell, or for a group of cells such as a tumor or biopsy sample. Gene expression data and gene expression levels can be stored on computer readable media, for example, the computer readable medium used in conjunction with a microarray or chip reading device. Such gene expression data can be manipulated to generate gene expression signatures.

The expression level of a biomarker gene can be determined using a reagent such as a probe, primer or antibody and/or a method performed on a tumor sample, for example a tumor sample of the subject, for ascertaining or measuring quantitatively, semi-quantitatively or qualitatively the amount of a of a polypeptide or mRNA (or cDNA derived therefrom) corresponding to one or more biomarker genes. For example, a level of a biomarker gene can be determined by a number of methods including for example immunoassays including for example immunohistochemistry, ELISA, Western blot, immunoprecipitation and the like, where a detection agent such as an antibody for example, a labeled antibody, specifically binds the encoded polypeptide and permits relative or absolute ascertaining of the amount of polypeptide encoded by the biomarker gene, hybridization and PCR protocols where a probe or primer or primer set are used to ascertain the amount of nucleic acid corresponding to the biomarker gene, including for example probe based and amplification based methods including for example microarray analysis, RT-PCR such as quantitative RT-PCR (qRT-PCR), gRT-PCR, serial analysis of gene expression (SAGE), Northern Blot, digital molecular barcoding technology, for example Nanostring Counter Analysis, and TaqMan quantitative PCR assays.

Other methods of mRNA detection and quantification can be applied, such as mRNA in situ hybridization in formalin-fixed, paraffin-embedded (FFPE) tissue samples or cells, which uses probe sets for each mRNA that bind specifically to an amplification system to amplify the hybridization signals; these amplified signals can be visualized using a standard fluorescence microscope or imaging system.

TaqMan probe-based gene expression analysis (PCR- based) can also be used for measuring biomarker gene expression levels in tissue samples, including mRNA levels in FFPE samples. TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded, and signals are measured by a detection system; these signal intensities are used to calculate the abundance of a given transcript (gene expression) in a sample.

As used herein, a “nucleic acid” can generally refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (for example, altered backbone, sugar, or nucleobase). “Nucleic acid”, “polynucleotide, “target polynucleotide”, and “target nucleic acid” can be used interchangeably.

As used herein, the term “mRNA” or sometimes refer by “mRNA transcripts” include but is not limited to pre-mRNA transcript(s), transcript processing intermediates, mature mRNA(s) ready for translation and transcripts of the gene or genes, or nucleic acids derived from the mRNA transcript(s). Transcript processing can include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

As used herein “sequencing” a nucleic acid molecule means determining the identity of at least one nucleotide in the molecule. In certain embodiments, the identity of less than all the nucleotides in a molecule is determined. In other embodiments, the identity of a majority or all the nucleotides in the molecule is determined.

As used herein, the term “tumor sample” refers to any sample containing tumor material obtained from a subject. A tumor sample can be obtained from a subject prior to or after a diagnosis, at one or more time points prior to or following treatment or therapy, at one or more time points during which there is no treatment or therapy or can be collected from a healthy subject. The tumor sample can be a tissue sample or a fluid sample. In certain embodiments, the tumor sample includes a tissue sample, a biopsy sample, a tumor aspirate, a bone marrow aspirate, or a blood sample (or a fraction thereof, such as blood or serum). In certain embodiments, the tumor sample includes a tumor cell or cancer cell, for example a circulating tumor cell present in a fluid sample, for example, blood or a fraction thereof. In certain embodiments, the tumor sample includes a cell free nucleic acid present in a fluid sample, for example, blood or a fraction thereof. In certain embodiments, the tumor sample comprises a cell lysate (or lysate fraction) or cell extract; or a solution containing one or more molecules derived from a cell or cellular material (for example a polypeptide or nucleic acid). The cell lysate can include proteins, nuclear and/or mitochondrial fractions. In certain embodiments, the cell lysate includes a cytosolic fraction. In some embodiments, the cell lysate includes a nuclear/mitochondrial fraction and a cytosolic fraction.

The source of a tumor sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid, or interstitial fluid; or cells from any time in gestation or development of the subject. The tumor sample can contain compounds that are not naturally intermixed with the tissue in nature such as preservatives, anticoagulants, buffers, fixatives, nutrients, antibiotics, or the like. The tumor sample can be preserved as a frozen sample or as formaldehyde- or paraformaldehyde-fixed paraffin-embedded (FFPE) tissue preparation. For example, the sample can be embedded in a matrix, for example, an FFPE block or a frozen sample. However, other tissue and sample types are amenable for use herein. In certain embodiments, the other tissue and sample types can be fresh frozen tissue, wash fluids, or cell pellets, or the like. A tumor sample can be a tumor sample, which contains nucleic acid molecules from a tumor or cancer. A tumor sample that is a tumor sample can be DNA, for example, genomic DNA, or cDNA derived from RNA. In certain embodiments, the tumor nucleic acid sample is purified or isolated (for example, it is removed from its natural state). In certain embodiments, the sample is a tissue (for example, a tumor biopsy), a CTC or cell free nucleic acid.

In certain embodiments, a tumor sample is obtained from a human subject. In a further embodiment, the analysis is performed on a tumor biopsy embedded in paraffin wax. In certain embodiments, the sample can be a fresh frozen tissue sample. In another embodiment, the sample can be a bodily fluid obtained from the subject. The bodily fluid can be blood or fractions thereof (specifically, serum, plasma), urine, saliva, sputum, or cerebrospinal fluid (CSF). The sample can contain cellular as well as extracellular sources of nucleic acid. The extracellular sources can be cell-free nucleic acids and/or exosomes. The methods described herein, including the RT-PCR methods, are sensitive, precise and have multi- analyte capability for use with paraffin embedded samples. See, for example, Cronin et al., Am. J Pathol. 164(1):35-42 (2004).

General methods for mRNA extraction are well known in the art and are disclosed in standard textbooks of molecular biology, including Ausubel et al, ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers according to the manufacturers’ instructions. RNA prepared from a tumor can be isolated, for example, by cesium chloride density gradient centrifugation.

“Likely to” or “increased likelihood,” as used herein, refers to an increased probability that an event will occur. Thus, in one example, a subject that is likely to respond to treatment with a SHP2 inhibitor has an increased probability of responding to treatment with the SHP2 inhibitor relative to a reference subject or group of subjects.

“Unlikely to” refers to a decreased probability that an event, item, object, thing or person will occur with respect to a reference. Thus, a subject that is unlikely to respond to treatment with a SHP2 inhibitor has a decreased probability of responding to treatment with the SHP2 inhibitor relative to a reference subject or group of subjects.

As used herein, “genomic profiling” means sequencing a part or all of the genome of a subject, such as to identify the nucleotide sequence of one or more genes in the subject, such as to identify genomic alterations (for example, mutations) in one or more biomarker genes that would identify the subject as a candidate to receive certain drugs or other therapeutic agents. Genomic profiling can be performed by a method described herein, such as by a next-generation sequencing method, or a massively parallel sequencing method.

The term “probe” refers to any molecule which is capable of selectively binding to a specifically intended target molecule, for example, a nucleotide transcript or protein encoded by or corresponding to a marker. Probes can be either synthesized by one skilled in the art or derived from appropriate tumor preparations. For purposes of detection of the target molecule, probes can be specifically designed to be labeled, as described herein. Examples of molecules that can be utilized as probes include, but are not limited to, RNA, DNA, proteins, antibodies, and organic molecules.

As used herein, the term “genotype” refers to the alleles at one or more specific biomarker genes. The genotype of a biomarker gene can be determined by methods that include nucleic acids (RNA, cDNA, and DNA) and proteins, and variants and fragments thereof.

As used herein, the term “sensitive” in the context of a SHP2 inhibitor therapy, means that the SHP2 inhibitor therapy is more effective at reducing the tumor relative to a control drug in a subject of the same genotype.

As used herein, the term “resistant” in the context of a SHP2 inhibitor therapy, means means that the SHP2 inhibitor therapy is less effective at reducing the tumor relative to a control drug in a subject of the same genotype.

As described herein, responses to a SHP2 inhibitor include sensitivity and resistance compared to the response to a control drug in a subject of the same genotype. Such genotype-specific therapeutic responses (GSTRs) that can be characterized based on the relative numbers of tumors above a certain size after treatment (ScoreRTN - Relative Tumor Number) and the geometric mean of tumors from the full distribution of tumor sizes (ScoreRGM - Relative Geometric Mean) as described in Li, C., Lin, W.-Y et al. Quantitative in vivo analyses reveal a complex pharmacogenomic landscape in lung adenocarcinoma. biorxiv, 2020.01.28.923912 (doi.org/10.1101/2020.01.28.923912)

In certain embodiments, the resistance and/or sensitivity profiles of one or more biomarker genes for a SHP2 inhibitor can be compared to the corresponding resistance and/or sensitivity scores for a standard of care therapy in order to determine whether a subject is likely to benefit from SHP2 inhibitor therapy. For example, SHP2 inhibitor therapy can be compared to standard of care (SoC) therapy for a particular cancer to determine which genotypes are sensitive or resistant relative to the SoC therapy using the described herein. Then, by excluding resistant patients and enrichment for sensitive patients, the performance of a SHP2 inhibitor relative to SoC can be improved.

In certain embodiments, the resistance and/or sensitivity profiles of one or more biomarker genes for a SHP2 inhibitor can be compared to the corresponding resistance and/or sensitivity scores for a standard of care therapy in order to determine whether a subject is likely to benefit from SHP2 inhibitor therapy. For example, if there are four biomarker genes predictive of resistance to SHP2 inhibitor therapy and two of the four biomarker genes show a lower resistance to SHP2 inhibitor therapy relative to the SoC therapy, while the other two show a higher resistance relative to the standard of care therapy, only the former two biomarker genes can be used for selecting the subject for SHP2 inhibitor therapy over the standard of care therapy.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A nucleic acid molecule corresponding to a biomarker gene of the present present disclosure can be isolated using standard molecular biology techniques and the sequence information in the database records described herein. Using all or a portion of such nucleic acid sequences, nucleic acid molecules of the present present disclosure can be isolated using standard hybridization and cloning techniques (for example, as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the present present disclosure can be amplified using cDNA, mRNA, or genomic DNA as a template and appropriate oligonucleotide primers according to standard PCR amplification techniques.

As used herein, the terms “peptide,” “polypeptide,” or “protein” can refer to a molecule comprised of amino acids and can be recognized as a protein by those of ordinary skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In the case of measuring protein levels to determine biomarker gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent, or other detectable reagents to facilitate detection. Methods of detection include, without limitation, enzyme-linked immunosorbent assay (ELISA) and immunoblot techniques.

In some embodiments, the methods described herein include determining the genotype of one or more biomarker genes such as APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3. In certain embodiments, the one or more biomarker genes comprise EP300, FBXW7, KMT2D, NF1, RB1, RNF43, SETD2, and SMAD4. In certain embodiments, the one or more biomarker genes comprise ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2. In certain embodiments, the one or more biomarker genes comprise ARID1A, BAP1, CREBBP, DICER1, KDM6A, KEAP1, KRASG12D, PALB2, and SMARCA4. In certain embodiments, the one or more biomarker genes comprise ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4. In certain embodiments, the one or more biomarker genes comprise BAP1, CUL3, DICER1, KDM6A, KRAS, NCOA6, NF2, PTEN, SMARCA4, TP53, TSC1, and TSC2. In certain embodiments, the one or more biomarker genes comprise EP300, FBXW7, and RB1. In certain embodiments, the one or more biomarker genes comprise CREBBP, PBRM1, and SMG1. In certain embodiments, the one or more biomarker genes comprise PBRM1. In certain embodiments, the one or more biomarker genes comprise KEAP1.

In certain embodiments, the present disclosure provides a method of predicting resistance of tumor growth to inhibition by a therapy comprising a SHP2 inhibitor, comprising: (a) detecting in a tumor sample of a subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of ARID1A, BAP1, CREBBP, DICER1, KDM6A, KEAP1, KRASG12D, PALB2, and SMARCA4 (ii) a decreased copy number of one or more of ARID1A, BAP1, CREBBP, DICER1, KDM6A, KEAP1, KRASG12D, PALB2, or SMARCA4, or (iii) a decreased expression of mRNA or protein in or more of ARID1A, BAP1, CREBBP, DICER1, KDM6A, KEAP1, KRASG12D, PALB2, and SMARCA4.

A method of predicting resistance of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4 (ii) a decreased copy number of one or more of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4, or (iii) a decreased expression of mRNA or protein in or more of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4.

In certain embodiments, the present disclosure provides a method of predicting resistance of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of BAP1, CUL3, DICER1, KDM6A, KRAS, NCOA6, NF2, PTEN, SMARCA4, TP53, TSC1, and TSC2 ; (ii) a decreased copy number of one or more of BAP1, CMJ, DICER1, KDM6A, KRAS, NCOA6, NF2, PTEN, SMARCA4, TP53, TSC1, and TSC2, or (iii) a decreased expression of mRNA or protein in or more of BAP1, CUL3, DICER1, KDM6A, KRAS, NCOA6, NF2, PTEN, SMARCA4, TP53, TSC1, and TSC2.

In certain embodiments, the present disclosure provides a method of predicting resistance of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in one or more of CREBBP, PBRM1, and SMG1; (ii) a decreased copy number of one or more of CREBBP, PBRM1, and SMG1, or (iii) a decreased expression of mRNA or protein in or more of CREBBP, PBRM1, and SMG.

In certain embodiments, the present disclosure provides a method of predicting resistance of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting resistance of tumor cell growth to inhibition by the therapy comprising a SHP2 inhibitor, if the tumor sample comprises (i) an inactivating mutation in PBRM1; (ii) a decreased copy number of PBRM1, or (iii) a decreased expression of mRNA or protein of PBRM1.

In some embodiments, the present disclosure provides a method of enriching a prospective patient population for subjects likely to respond to a SHP2 inhibitor therapy, comprising performing the methods of predicting resistance of tumor growth to inhibition by a therapy comprising a SHP2 inhibitor, as described herein, on two or more individual subjects within the prospective patient population.

A method of predicting sensitivity of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in one or more of EP300, FBXW7, KMT2D, NF1, RB1, RNF43, SETD2, and SMAD4, (ii) a decreased copy number of one or more of EP300, FBXW7, KMT2D, NF1, RB1, RNF43, SETD2, and SMAD4, or (iii) decreased expression of mRNA or protein in one or more of EP300, FBXW7, KMT2D, NF1, RB1, RNF43, SETD2, and SMAD4.

A method of predicting sensitivity of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in one or more of EP300, FBXW7, and RB1, (ii) a decreased copy number of one or more of EP300, FBXW7, and RB1, or (iii) decreased expression of mRNA or protein in one or more of EP300, FBXW7, and RB1.

A method of predicting sensitivity of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in one or more of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2, (ii) a decreased copy number of one or more of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2, or (iii) decreased expression of mRNA or protein in one or more of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2.

A method of predicting sensitivity of tumor growth to inhibition by a therapy comprising a SHP2 inhibitor, comprising: (a) detecting in a tumor sample ofa subject a genotype of one or more biomarker genes; (b) analyzing the genotype of the one or more biomarker genes in the tumor sample, and (c) predicting sensitivity of tumor cell growth to inhibition by the therapy comprising a SHP2 inhibitor, if the tumor sample comprises (i) an inactivating mutation in KEAP1, (ii) a decreased copy number of KEAP1, or (iii) decreased expression of mRNA or protein of KEAP1.

In some embodiments, the present disclosure provides a method of enriching a prospective patient population for subjects likely to respond to a SHP2 inhibitor therapy, comprising performing the methods of predicting sensitivity of tumor growth to inhibition by a therapy comprising a SHP2 inhibitor, as described herein, on two or more individual subjects within the prospective patient population.

A method of predicting response of tumor growth to inhibition by a therapy comprising a SHP2 inhibitor, comprising: (a) determining in a tumor sample ofa subject a genotype of one or more biomarker genes; (b) analyzing the genotype of the one or more biomarker genes in the tumor sample, and (c) predicting the response of tumor cell growth to inhibition by the therapy comprising a SHP2 inhibitor, if the tumor sample comprises (i) an inactivating mutation in one or more of ARID1A, BAP1, CREBBP, DICER1, KDM6A, KEAP1, KRASG12D, PALB2, and SMARCA4 (ii) a decreased copy number of one or more of ARID1A, BAP1, CREBBP, DICER1, KDM6A, KEAP1, KRASG12D, PALB2, and SMARCA4, or (iii) a decreased expression of mRNA or protein in or more of ARID1A, BAP1, CREBBP, DICER1, KDM6A, KEAP1, KRASG12D, PALB2, and SMARCA4; (iv) an inactivating mutation in one or more of EP300, FBXW7, KMT2D, NF1, RB1, RNF43, SETD2, and SMAD4, (v) a decreased copy number of one or more of EP300, FBXW7, KMT2D, NF1, RB1, RNF43, SETD2, and SMAD4, or (vi) a decreased expression of mRNA or protein in one or more of EP300, FBXW7, KMT2D, NF1, RB1, RNF43, SETD2, and SMAD4.

A method of predicting response of tumor growth to inhibition by a therapy comprising a SHP2 inhibitor, comprising: (a) determining in a tumor sample ofa subject a genotype of one or more biomarker genes; (b) analyzing the genotype of the one or more biomarker genes in the tumor sample, and (c) predicting the response of tumor cell growth to inhibition by the therapy comprising a SHP2 inhibitor, if the tumor sample comprises (i) an inactivating mutation in one or more of BAP1, CUL3, DICER1, KDM6A, KRAS, NCOA6, NF2, PTEN, SMARCA4, TP53, TSC1, and TSC2; (ii) a decreased copy number of one or more of BAP1, CUL3, DICER1, KDM6A, KRAS, NCOA6, NF2, PTEN, SMARCA4, TP53, TSC1, and TSC2, or (iii) a decreased expression of mRNA or protein in or more of BAP1, CUL3, DICER1, KDM6A, KRAS, NCOA6, NF2, PTEN, SMARCA4, TP53, TSC1, and TSC2; (iv) an inactivating mutation in one or more of EP300, FBXW7, and RB1, (v) a decreased copy number of one or more of EP300, FBXW7, and RB1, or (vi) a decreased expression of mRNA or protein in one or more of EP300, FBXW7, and RB1.

A method of predicting response of tumor growth to inhibition by a therapy comprising a SHP2 inhibitor, comprising: (a) determining in a tumor sample ofa subject a genotype of one or more biomarker genes; (b) analyzing the genotype of the one or more biomarker genes in the tumor sample, and (c) predicting the response of tumor cell growth to inhibition by the therapy comprising a SHP2 inhibitor, if the tumor sample comprises (i) an inactivating mutation in one or more CREBBP, PBRM1, and SMG1; (ii) a decreased copy number of one or more of CREBBP, PBRM1, and SMG1, or (iii) a decreased expression of mRNA or protein in or more of CREBBP, PBRM1, and SMG1; (iv) an inactivating mutation in KEAP1, (v) a decreased copy number of KEAP1, or (vi) a decreased expression of mRNA or protein in KEAP1.

In some embodiments, the present disclosure provides a method of enriching a prospective patient population for subjects likely to respond to a SHP2 inhibitor therapy, comprising performing the methods of predicting response of tumor growth to inhibition by a therapy comprising a SHP2 inhibitor, as described herein, on two or more individual subjects within the prospective patient population.

In some embodiments, the methods provide a further step of treating cancer in the subject based on the predicted response of tumor growth to inhibition by a therapy comprising a SHP2 inhibitor. In some embodiments, the methods comprise treating the subject with a therapy comprising a SHP2 inhibitor.

In some embodiments, the methods comprise treating the subject with a therapy other than a SHP2 inhibitor therapy, for example and without limitation, a combination chemotherapy, a combination chemotherapy and a targeted therapy with a monoclonal antibody, such as bevacizumab, cetuximab, or necitumumab; a combination chemotherapy followed by a second chemotherapy; a targeted therapy with an EGFR tyrosine kinase inhibitor, such as osimertinib, dacomitinib, gefitinib, erlotinib, or afatinib; targeted therapy with an ALK inhibitor, such as alectinib, lorlatinib, crizotinib, ceritinib, or brigatinib; a targeted therapy with a BRAF inhibitor or a MEK inhibitor, such as dabrafenib or trametinib; targeted therapy with crizotinib and entrectinib to stop proteins from being made by the ALK and ROS1 genes; targeted therapy with a NTRK inhibitor, such as larotrectinib or entrectinib; a targeted therapy with a RET inhibitor, such as selpercatinib; a targeted therapy with a MET inhibitor, such as tepotinib or capmatinib; immunotherapy with an immune checkpoint inhibitor, such as pembrolizumab or atezolizumab, with or without chemotherapy; a targeted therapy with an mTOR inhibitor, such as everolimus; laser therapy and/or internal radiation therapy for tumors that are blocking the airways; external radiation therapy; surgery, and clinical trials of new drugs and combinations of treatments.

In some embodiments, the present disclosure provides a method of treating cancer in a subject, comprising treating a subject with a SHP2 inhibitor when a tumor sample of the subject comprises (i) an inactivating mutation in one or more of CDKN2A, EP300, KRAS, MGA, EP300, FBXW7, or RB1, (ii) a decreased copy number of one or more of EP300, FBXW7, or RB1, or (iii) decreased expression of mRNA or protein in one or more of EP300, FBXW7, or RB1. In some embodiments, the tumor sample ofthe subject further comprises absence of (i) an inactivating mutation in one or more of BAP1, CUL3, DICER1, KDM6A, KRAS, NCOA6, NF2, PTEN, SMARCA4, TP53, TSC1, or TSC2, (ii) a decreased copy number of one or more of BAP1, CUL3, DICER1, KDM6A, KRAS, NCOA6, NF2, PTEN, SMARCA4, TP53, TSC1, or TSC2, or (iii) decreased expression of BAP1, CUL3, DICER1, KDM6A, KRAS, NCOA6, NF2, PTEN, SMARCA4, TP53, TSC1, or TSC2 mRNA or protein.

In some embodiments, the present disclosure provides a method of treating cancer in a subject, comprising treating a subject with a SHP2 inhibitor when a tumor sample ofthe subject comprises (i) an inactivating KEAP1 mutation, (ii) a decreased copy number of KEAP1, or (iii) decreased expression of KEAP1 mRNA or protein. In some embodiments, the tumor sample ofthe subject further comprises absence of (i) an inactivating mutation in one or more of CREBBP, PBRM1, or SMG1, (ii) a decreased copy number of one or more of CREBBP, PBRM1, or SMG1, or (iii) a decreased expression of CREBBP, PBRM1, or SMG1 mRNA or protein. In some embodiments, the tumor sample ofthe subject further comprises absence of (i) an inactivating mutation in PBRM1, (ii) a decreased copy number of PBRM1, or (iii) a decreased expression of PBRM1 mRNA or protein.

In some embodiments, the present disclosure provides a composition comprising one or more isolated biomarker genes selected from the group consisting of ARID1A, BAP1, CREBBP, DICER1, KDM6A, KEAP1, KRASG12D, PALB2, and SMARCA4. In some embodiments, the present disclosure provides a composition comprising one or more isolated biomarker genes selected from the group consisting of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4. In some embodiments, the present disclosure provides a composition comprising one or more isolated biomarker genes selected from the group consisting of CREBBP, PBRM1, and SMG1 In some embodiments, the present disclosure provides a composition comprising one or more isolated biomarker genes selected from the group consisting of EP300, FBXW7, and RB1. In some embodiments, the present disclosure provides a composition comprising one or more isolated biomarker genes selected from the group consisting of EP300, FBXW7, KMT2D, NF1, RB1, RNF43, SETD2, and SMAD4. In some embodiments, the present disclosure provides a composition comprising one or more isolated biomarker genes selected from the group consisting of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2.

In some embodiments, the present disclosure provides a method of detecting one or more isolated biomarker genes selected from the group consisting of ARID1A, BAP1, CREBBP, DICER1, KDM6A, KEAP1, KRASG12D, PALB2, and SMARCA4 in a subject, comprising detecting whether the one or more isolated biomarker genes are present in a tumor sample of a subject by contacting the tumor sample with a binding agent and detecting binding between the one or more isolated biomarker genes and the binding agent. In certain embodiments, the method further comprises obtaining a tumor sample from the subject.

In some embodiments, the present disclosure provides a method of detecting one or more isolated biomarker genes selected from the group consisting of ARJD1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4 in a subject, comprising detecting whether the one or more isolated biomarker genes are present in a tumor sample of a subject by contacting the tumor sample with a binding agent and detecting binding between the one or more isolated biomarker genes and the binding agent. In certain embodiments, the method further comprises obtaining a tumor sample from the subject.

In some embodiments, the present disclosure provides a method of detecting one or more isolated biomarker genes selected from the group consisting of BAP1, CUL3, DICER1, KDM6A, KRAS, NCOA6, NF2, PTEN, SMARCA4, TP53, TSC1, and TSC2 in a subject, comprising detecting whether the one or more isolated biomarker genes are present in a tumor sample of a subject by contacting the tumor sample with a binding agent and detecting binding between the one or more isolated biomarker genes and the binding agent. In certain embodiments, the method further comprises obtaining a tumor sample from the subject.

In some embodiments, the present disclosure provides a method of detecting one or more isolated biomarker genes selected from the group consisting of CREBBP, PBRM1, and SMG1 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the method further comprises obtaining a tumor sample from the subject.

In some embodiments, the present disclosure provides a method of detecting one or more isolated biomarker genes selected from the group consisting of EP300, FBXW7, and RB1 in a subject, comprising detecting whether the one or more isolated biomarker genes are present in a tumor sample of the subject by contacting the tumor sample with a binding agent and detecting binding between the one or more isolated biomarker genes and the binding agent. In certain embodiments, the method further comprises obtaining a tumor sample from the subject.

In some embodiments, the present disclosure provides a method of detecting one or more isolated biomarker genes selected from the group consisting of EP300, FBXW7, KMT2D, NF1, RB1, RNF43, SETD2, and SMAD4 in a subject, and (b) detecting whether the one or more isolated biomarker genes are present in a tumor sample of the subject by contacting the tumor sample with a binding agent and detecting binding between the one or more isolated biomarker genes and the binding agent. In certain embodiments, the method further comprises obtaining a tumor sample from the subject.

In some embodiments, the present disclosure provides a method of detecting one or more isolated biomarker genes selected from the group consisting of ATRX, CDKN2A, DLC1, EP300, FBXW7, KMT2D, NF1, PBRM1, PTPRD, RASA1, RB1, RBM10, RNF43, SETD2, SMAD4, STK11, TET2, and TGFBR2 in a subject, and (b) detecting whether the one or more isolated biomarker genes are present in a tumor sample of the subject by contacting the tumor sample with a binding agent and detecting binding between the one or more isolated biomarker genes and the binding agent. In certain embodiments, the method further comprises obtaining a tumor sample from the subject.

In some embodiments, the subject is classified as sensitive or resistant to a therapy comprising a SHP2 inhibitor based on the methods of detecting one or more isolated biomarker genes described herein. Accordingly, in some embodiments, the methods of the present disclosure described herein further comprise classifying a subject as sensitive or resistant to a therapy comprising a SHP2 inhibitor.

In some embodiments, the present disclosure provides a method of determining effectiveness of a SHP2 inhibitor in reducing tumor size, comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; € comparing sizes of the first and second inert tumors after the therapy; (f) comparing sizes of the first and second mutant tumors after completion of the therapy, and (g) identifying the mutant tumor genotype as sensitive to the SHP2 inhibitor if the change in tumor size between the first and second inert tumors after the therapy is less than the change in tumor size between the first and second mutant tumors after the therapy.

A method of determining effectiveness of a SHP2 inhibitor in reducing tumor size, comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotype€(e) comparing sizes of the first and second inert tumors after the therapy; (f) comparing sizes of the first and second mutant tumors after completion of the therapy, and (g) identifying the mutant tumor genotype as resistant to the SHP2 inhibitor if the change in tumor size between the first and second inert tumors after the therapy is greater than the change in tumor size between the first and second mutant tumors after the therapy.

In some embodiments, the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma and cholangiocarcinoma. In some embodiments, the cancer is non-small cell lung cancer (NSCLC).

In some embodiments, the cancer is a KRAS-mutant cancer.

In some embodiments, the tumor sample comprises a mutation in at least one gene. In some embodiments, the tumor suppressor gene TP53. In some embodiments, the tumor sample has been previously genotyped and determined to comprise a mutation in at least one gene. In some embodiments, the gene is an oncogene. In some embodiments, the the oncogene is KRAS. In some embodiments, the gene is a tumor suppressor gene. In some embodiments, the tumor suppressor gene TP53. In some embodiments, the methods of the present disclosure described herein further comprise administering a SHP2 inhibitor therapy to the subject.

In some embodiments, the methods of the present disclosure described herein further comprise administering to the subject a second therapy. In some embodiments, the second therapy comprises an agent that affects KRAS activity between inactive guanosine diphosphate (GDP)-bound and active guanosine triphosphate (GTP)-bound states. In some embodiments, the second therapy comprises an agent that targets the immune system directly.

The present present disclosure provides, in part, methods for accurately classifying a subject afflicted with cancer as likely to be sensitive for therapy with a SHP2 inhibitor. The methods comprise determining a genotype of one or more biomarker genes in a tumor sample of the subject. In certain embodiments, the tumor sample (for example, tumor sample) comprises polypeptides encoded by the one or more biomarker genes. Alternatively, the tumor sample can comprise mRNA molecules or genomic DNA corresponding to the one or more biomarker genes. In some embodiments, the methods comprise contacting a tumor sample of the subject with a reagent capable of determining the genotype by detecting, for example, a polypeptide or nucleic acid that encodes the biomarker gene or fragments thereof.

The methods of the present disclosure detect mRNA, polypeptide, genomic DNA, or fragments thereof, in a tumor sample in vitro as well as in vivo. For example, in vitro techniques for detection of mRNA or a fragment thereof include Northern hybridizations and in situ hybridizations. In vitro techniques for detection of polypeptide include enzyme linked immunosorbent assays (ELISAs), Western blots, immunoprecipitations, and immunofluorescence. In vitro techniques for detection of biomarker genomic DNA or a fragment thereof include Southern hybridizations. Furthermore, in vivo techniques for detection of one or more polypeptides or fragments thereof include labeled antibodies. For example, the antibody can be labeled with a radioactive marker whose presence and location in a subject can be detected by standard imaging techniques.

In some embodiments, the genotype, presence or level of at least one, two, three, four, five, six, seven, eight, nine, ten, fifty, sixty, or more biomarker genes of the present disclosure is determined in the tumor sample. In some embodiments, methods of the present disclosure employ a statistical algorithm and/or empirical data (for example, the presence or level of one or biomarker genes described herein). In certain instances, a single learning statistical classifier system can be used to classify a sample. The use of a single learning statistical classifier system typically classifies the sample accurately with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of ordinary skill in the art. For example, learning statistical classifier systems include a machine learning algorithmic technique capable of adapting to complex data sets (for example, panel of markers of interest) and making decisions based upon such data sets. In some embodiments, a single learning statistical classifier system such as a classification tree (for example, random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (for example, decision/classification trees such as random forests, classification and regression trees (C&RT), boosted trees, etc.), Probably Approximately Correct (PAC) learning, connectionist learning (for example, neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (for example, passive learning in a known environment such as naive learning, adaptive dynamic learning, and temporal difference learning, passive learning in an unknown environment, active learning in an unknown environment, learning action-value functions, applications of reinforcement learning, and genetic algorithms and evolutionary programming. Other learning statistical classifier systems include support vector machines (for example, Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present disclosure further comprises sending the cancer classification results to a clinician, for example, an oncologist or hematologist.

In certain embodiments, determining the genotype of a biomarker gene comprises genomic profiling to directly determine the genotype of the one or more biomarker genes. In certain embodiments, genomic profiling comprises contacting the tumor sample with reagents, including, probes and/or primers, for sequencing a biomarker gene or portion thereof. In certain embodiments, probes or primers can be designed to detect a mutation in a biomarker gene. In certain embodiments, the mutation is an inactivating mutation. In certain embodiments, the mutation results in decreased gene expression.

In certain embodiments, the methods include directly determining the genotype of a biomarker gene by genomic profiling to detect the presence or absence of a genetic alteration characterized by at least one alteration affecting the integrity of a gene encoding one or more biomarkers polypeptide, or the mis-expression of the biomarker (for example, mutations and/or splice variants). For example, such genetic alterations can be detected by ascertaining the existence of at least one of a deletion of one or more nucleotides from one or more biomarker genes; an addition of one or more nucleotides to one or more biomarker genes; a substitution of one or more nucleotides of one or more biomarker genes; a chromosomal rearrangement of one or more biomarker genes; an alteration in the level of a mRNA transcript of one or more biomarker genes; aberrant modification of one or more biomarker genes; such as of the methylation pattern of the genomic DNA; the presence of a non-wild type splicing pattern of a messenger RNA transcript of one or more biomarker genes; a non-wild type level of one or more biomarkers polypeptide; allelic loss of one or more biomarker genes, and inappropriate post-translational modification of one or more biomarkers polypeptide. As described herein, there are a large number of assays known in the art which can be used for detecting alterations in one or more biomarker genes.

In certain embodiments, detection of the alteration involves the use of a probe/primer in a polymerase chain reaction (PCR), such as anchor PCR or RACE PCR, or, alternatively, in a ligation chain reaction (LCR) (see, for example, Landegran et al. (1988) Science 241:1077-1080; and Nakazawa et al. (1994) Proc. Natl. Acad. Sci. USA 91 :360-364), the latter of which can be particularly useful for detecting point mutations in one or more biomarker genes (see Abravaya et al. (1995) Nucleic Acids Res. 23:675-682). This method can include the steps of collecting a sample of cells from a subject, isolating nucleic acid from the cells of the sample, contacting the nucleic acid sample with one or more primers which specifically hybridize to one or more biomarker genes of the present disclosure, or fragments thereof, under conditions such that hybridization and amplification of the biomarker gene (if present) occurs, and detecting the presence or absence of an amplification product, or detecting the size of the amplification product and comparing the length to a control sample. It is anticipated that PCR and/or LCR can be desirable to use as a preliminary amplification step in conjunction with any of the techniques used for detecting mutations described herein.

Alternative amplification methods include self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of ordinary skill in the art. These detection schemes are especially useful for the detection of nucleic acid molecules if such molecules are present in very low numbers.

In certain embodiments, mutations in one or more biomarker genes of the present disclosure, or a fragment thereof, from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

In other embodiments, genetic mutations in one or more biomarker genes of the present disclosure, or a fragment thereof, can be identified by hybridizing a nucleic acid to high density arrays containing hundreds or thousands of oligonucleotide probes (Cronin, M. T. et al. (1996) Hum. Mutat. 7:244-255; Kozal, M. J. et al. (1996) Nat. Med. 2:753-759).

In certain embodiments, any of a variety of sequencing methods known in the art can be used to directly sequence one or more biomarker genes of the present disclosure, or a fragment thereof, and detect mutations by comparing the sequence of the sample biomarker gene with the corresponding wild-type (control) sequence. Examples of sequencing reactions include next-generation sequencing to determine the nucleotide sequence of either individual nucleic acid molecules (for example, in single molecule sequencing) or clonally expanded proxies for individual nucleic acid molecules in a highly parallel fashion. Next generation sequencing methods are known in the art, and are described, for example, in Metzker, M. (2010) Nature Biotechnology Reviews 11:31-46. Next generation sequencing collectively refers to several DNA/RNA sequencing technologies that vary according to the input material, length of read, and portion of the genome to be sequenced. Broadly, the two major next generation sequencing technologies are short-read sequencing and long-read sequencing. Short-read sequencing generally refers to reads that are shorter than 300 bp, whereas long-read sequencing refers to reads that are longer than 2.5 Kb. Short-read sequencing is a relatively inexpensive option (low costs per Gb) that has a high level of accuracy and is used more frequently in clinical practice for the detection of specific mutation hotspots. Moreover, based on the initial input material, different sequencing approaches can be used (for example, genomic DNA [DNA-seq], messenger or noncoding RNA [RNA-seq], or any nucleic or ribonucleic material obtained following the use of certain procedures).

Current next generation sequencing approaches also differ based on the extent of target enrichment and sequencing involved, with the 3 major types being whole genome sequencing (WGS), whole-exome sequencing (WES), and targeted gene panels. WGS refers to sequencing the entire genome, including coding and noncoding regions. It allows detection of several types of genetic aberrations, including single nucleotide variants and/or such structural alterations as insertions or deletions (also called indels), copy number variations involving duplications or deletions of long stretches of a chromosomal region, and rearrangements involving gross alterations in chromosomes or large chromosomal regions. WES involves sequencing only the coding regions of the genome and is limited in its ability to detect rearrangements between genes with breakpoints that frequently occur in intronic regions. RNA-based whole-transcriptome approaches can be another strategy to identify gene rearrangements.

Another next generation sequencing strategy, which is currently the most used approach to cancer genotyping in clinical use, is targeted gene panels that interrogate a discrete number of genes. This approach has the advantage of being able to focus on clinically relevant targets with deeper sequencer and focused analyses. Targeted gene panels can be performed with either amplicon-based or hybrid-capture enrichment strategies and can range from small, hotspot-only panels focusing on less than fifty genes to larger, more comprehensive panels that include hundreds to greater than a thousand genes with selected intronic tiling coverage. In addition to lower costs, the advantages of targeted gene panels include greater analytic sensitivity because of the greater depth of coverage, less complex data analysis and interpretation than would be necessary for WES and WGS, and greater flexibility that allows for tailoring the testing to genomic regions relevant to cancer. Any of the known Next generation sequencing approaches can be practiced for the methods described herein and a skilled person will be able to select the best sequencing strategy to practice the methods described herein.

In certain embodiments, determining the genotype of a biomarker gene comprises measuring the expression level of one or more biomarker genes. The expression level can be measured in several ways, including, but not limited to measuring the mRNA encoded by the biomarker genes; measuring the amount of protein encoded by the biomarker genes; and measuring the activity of the protein encoded by the biomarker genes. In some embodiments, a genotype of a biomarker gene is determined by measuring RNA, cDNA, protein or any combination thereof. When a genotype is determined by measuring RNA, the RNA can be reverse transcribed to produce cDNA (such as by RT-PCR), and the produced cDNA expression level can be detected. The expression level of a biomarker gene can be detected by forming a complex between a nucleic acid corresponding to a biomarker gene and a labeled probe or primer. When the nucleic acid is RNA or cDNA, the RNA or cDNA can be detected by forming a complex between the RNA or cDNA and a labeled nucleic acid probe or primer. The complex between the RNA or cDNA and the labeled nucleic acid probe or primer can be a hybridization complex.

Another method of determining the genotype of a biomarker gene at the nucleic acid level is the use of an amplification method such as, for example, RT-PCR or quantitative RT-PCR (qRT- PCR). Methods for determining the level of mRNA in a sample can involve the process of nucleic acid amplification, for example, by RT-PCR, ligase chain reaction or any other nucleic acid amplification method, followed by the detection of the amplified molecules using techniques well known to those of ordinary skill in the art. Numerous different PCR or qRT-PCR protocols are known in the art and can be directly applied or adapted for use using the presently described compositions for the detection and/or quantification of expression of biomarker genes in a sample.

Isolated mRNA can be used in hybridization or amplification assays that include, but are not limited to, Southern or Northern analyses, PCR analyses and probe arrays. One method for the detection of mRNA levels involves contacting the isolated mRNA or synthesized cDNA with a nucleic acid molecule (probe) that can hybridize to the mRNA encoded by the gene being detected. The nucleic acid probe can be, for example, a cDNA, or a portion thereof, such as an oligonucleotide of at least about 7, about 15, about 30, about 50, about 100, about 250, or about 500 nucleotides in length and sufficient to specifically hybridize under stringent conditions to the non-natural cDNA or mRNA.

As described herein, biomarker gene expression can be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular biomarker gene is characterized by a measure of gene transcript (for example mRNA), by a measure of the quantity of translated protein, or by a measure of gene product activity. Biomarker gene expression can be monitored in a variety of ways, including by detecting mRNA levels, protein levels, or protein activity, any of which can be measured using standard techniques. Detection can involve quantification of the level of gene expression (for example, genomic DNA, cDNA, mRNA, protein, or enzyme activity), or, alternatively, can be a qualitative assessment of the level of gene expression, in particular in comparison with a control level. The type of level being detected will be clear to the skilled person from the context.

In certain embodiments, detecting or determining expression levels of a biomarker gene and functionally similar homologs thereof, including a fragment or genetic alteration thereof (for example, in regulatory or promoter regions thereof) comprises detecting or determining RNA levels for the biomarker marker gene. In certain embodiments, one or more cells from the subject to be tested are obtained and RNA is isolated from the cells.

General methods for mRNA extraction are well known in the art and are disclosed in standard textbooks of molecular biology, including Ausubel et al, ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers according to the manufacturers’ instructions. RNA prepared from a tumor can be isolated, for example, by cesium chloride density gradient centrifugation.

The population of RNA can optionally be enriched, and further be amplified. For example, where RNA is mRNA, an amplification process such as RT-PCR can be utilized to amplify the mRNA, such that a signal is detectable, or detection is enhanced. Such an amplification process is beneficial particularly when the tumor, tissue, or tumor sample is of a small size or volume. Various amplification and detection methods can be used. For example, it is within the scope of the present disclosure to reverse transcribe mRNA into cDNA followed by polymerase chain reaction (RT-PCR); or, to use a single enzyme for both steps, or reverse transcribe mRNA into cDNA followed by symmetric gap ligase chain reaction (RT-AGLCR).

Many techniques are known in the state of the art for determining absolute and relative levels of gene expression, commonly used techniques suitable for use in the present disclosure include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed, and analyzed by autoradiography.

In situ hybridization visualization can also be employed, wherein a radioactively labeled antisense RNA probe is hybridized with a thin section of a biopsy sample, washed, cleaved with Rnase and exposed to a sensitive emulsion for autoradiography. The samples can be stained with hematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. Labeled nucleic acids of a test sample obtained from a subject can be hybridized to a solid surface comprising biomarker DNA. Positive hybridization signal is obtained with the sample containing biomarker transcripts. In certain embodiments, gene expression can be detected by microarray analysis. Differential gene expression can also be identified or confirmed using a microarray technique. The expression levels of one or more biomarker genes can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method. Quantitation of hybridization of each arrayed element allows for assessment of corresponding mRNA abundance. Microarray analysis can be performed by commercially available equipment, following manufacturer’s protocols.

Types of probes that can be used in the methods described herein include cDNA, riboprobes, synthetic oligonucleotides and genomic probes. The type of probe used will generally be dictated by the situation, such as riboprobes for in situ hybridization, and cDNA for Northern blotting, for example. In certain embodiments, the probe is directed to nucleotide regions unique to the RNA. The probes can be as short as is required to differentially recognize marker mRNA transcripts, and can be as short as, for example, about 15 bases; however, probes of at least 17, 18, 19 or 20 or more bases can be used. In certain embodiments, the primers and probes hybridize specifically under stringent conditions to a DNA fragment having the nucleotide sequence corresponding to the marker. As herein used, the term “stringent conditions” means hybridization will occur only if there is at least about 95% identity in nucleotide sequences. In certain embodiments, hybridization under “stringent conditions” occurs when there is at least 97% identity between the sequences.

The activity, level or presence of a protein encoded by a biomarker gene can be detected and/or quantified by detecting or quantifying the expressed polypeptide. The polypeptide can be detected and quantified by any of several means well known to those of ordinary skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (for example, Basic and Clinical Immunology, Sites and Terr, eds., Appleton and Lange, Norwalk, Conn. pp 217-262, 1991 which is incorporated by reference).

ELISA and RIA procedures can be conducted such that a desired protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, wherein a second antibody is used to bind the first, and radioactivity or the immobilized enzyme assayed (competitive assay). Alternatively, the protein in the sample is allowed to react with the corresponding immobilized antibody, radioisotope- or enzyme-labeled antibody is allowed to react with the system, and radioactivity or the enzyme assayed (ELISA-sandwich assay). Other conventional methods can also be employed as suitable.

Enzymatic and radiolabeling of a protein encoded by a biomarker gene and/or the antibodies can be affected by conventional means. It is possible to immobilize the enzyme itself on a support, but if solid-phase enzyme is required, then this is generally best achieved by binding to antibody and affixing the antibody to a support, models and systems for which are well-known in the art.

Other techniques can be used to detect protein corresponding to a biomarker gene according to a practitioner’s preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies specific for the protein (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection can also be used.

Immunohistochemistry can be used to detect expression of a protein corresponding to a biomarker gene, for example, in a biopsy sample. A suitable antibody is brought into contact with, for example, a thin layer of cells, washed, and then contacted with a second, labeled antibody. Labeling can be by fluorescent markers, enzymes, such as peroxidase, avidin, or radiolabelling. The assay is scored visually, using microscopy. Any other art-known method can be used to detect a protein corresponding to a biomarker gene.

### EXAMPLES OF NON-LIMITING ASPECTS OF THE DISCLOSURE

Aspects, including embodiments, of the present disclosure described above may be beneficial alone or in combination, with one or more other aspects or embodiments. Without limiting the foregoing description, certain non-limiting aspects of the present disclosure are provided below. As will be apparent to those of ordinary skill in the art upon reading the present disclosure, each of the individually numbered aspects may be used or combined with any of the preceding or following individually numbered aspects. This is intended to provide support for all such combinations of aspects and is not limited to combinations of aspects explicitly provided below:

1. A method for determining a genotype of one or more biomarker genes comprising APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP 15, and ZFHX3 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

2. The method of embodiment 1, further comprising (c) classifying the subject as sensitive or resistant to a therapy comprising a SH2 containing protein tyrosine phosphatase-2 inhibitor (SHP2i) based on the genotype of the one or more biomarker genes in the tumor sample of said subject.

3. The method of embodiment 1 or 2, wherein the biomarker panel comprises ARID1A, BAP1, CREBBP, DICER1, KDM6A, KEAP1, KRASG12D, PALB2, and SMARCA4.

4. The method of embodiment 3, wherein the biomarker panel further comprises ARID2, ASXL1, ATM, BRCA1, MTAP, NCOA6, NF2, PTEN, PTPN13, and PTPRS,

5. The method of embodiment 1 or 2, wherein the biomarker panel comprises BAP1, CUL3, DICER1, KDM6A, KRAS, NCOA6, NF2, PTEN, SMARCA4, TP53, TSC1, and TSC2

6. The method of embodiment 1 or 2, wherein the biomarker panel comprises EP300, FBXW7, and RB1.

7. The method of embodiment 6, wherein the biomarker panel further comprises KMT2D, NF1, RNF43, SETD2, and SMAD4.

8. The method of embodiment 7, wherein the biomarker panel further comprises ATRX, CDKN2A, DLC1, PBRM1, PTPRD, RASA1, RBM10, STK11, TET2, and TGFBR2.

9. The method of embodiment 1 or 2, wherein the biomarker panel comprises PBRM1.

10. The method of embodiment 9, wherein the biomarker panel further comprises CREBBP and SMG1.

11. The method of embodiment 1 or 2, wherein the biomarker panel comprises KEAP1.

12. The method of any one of embodiments 1-11, further comprising obtaining a tumor sample from the subject.

13. The method of any one of embodiments 1-12, wherein the genotype comprises a mutation in the one or more biomarker genes.

14. The method of embodiment 13, wherein the mutation inactivates the biomarker gene.

15. The method of any one of embodiments 1-14, further comprising comparing the genotype with a reference genotype.

16. The method of any one of embodiments 1-15, wherein the genotype is reported as a score.

17. The method of any one of embodiments 1-16, wherein determining the genotype comprises genomic profiling.

18. The method of any one of embodiments 1-16, wherein determining the genotype comprises measuring gene expression.

19. The method of embodiment 18, wherein measuring gene expression comprises detection of ribonucleic acids (RNAs) or polypeptides.

20. The method of any one of embodiments 2-19, wherein the subject is classified as sensitive to SHP2 inhibitor treatment.

21. The method of any one of embodiments 2-19, wherein the subject is classified as resistant to SHP2 inhibitor treatment.

22. The method of any one of embodiments 1-21, wherein the cancer is a KRAS-mutant cancer.

23. The method of any one of embodiments 1-22, wherein the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma, and cholangiocarcinoma.

24. The method of embodiment 23, wherein the cancer is colorectal cancer.

25. The method of embodiment 23, wherein the cancer is lung cancer.

26. The method of embodiment 25, wherein the lung cancer is non-small cell lung cancer (NSCLC).

27. The method of embodiment 26, wherein the NSCLC is lung adenocarcinoma.

28. The method of embodiment 20, further comprising administering to the subject a SHP2 inhibitor therapy after said genotype determination.

29. The method of any one of embodiments 2-28, wherein the SHP2 inhibitor comprises a small molecule.

30. The method of embodiment 29, wherein the SHP2 inhibitor is selected from the group consisting of ERAS-601 (Erasca), BBP-398, RMC-4630, RMC-4550, JAB-3068, JAB-3312, RLY-1971, PF-07284892, TNO-155, ET0038, HBI-2376, HS-10381, and SH3809.

31. The method of embodiment 28, further comprising administering to the subject a second therapy.

32. The method of embodiment 31, wherein the second therapy comprises an agent that affects KRAS activity between inactive guanosine diphosphate (GDP)-bound and active guanosine triphosphate (GTP)-bound states.

33. The method of embodiment 32, wherein the second therapy comprises an agent that targets the immune system directly.

34. The method of embodiment 33, wherein the second therapy comprises a programmed cell death protein 1 (PD-1) inhibitor or a colony stimulating factor 1 receptor (CSF-1R) inhibitor.

35. The method of embodiment 33, wherein the second therapy comprises an immune checkpoint inhibitor.

36. The method of embodiment 35, wherein the immune checkpoint inhibitor inhibits programmed cell death protein 1 (PD-1).

37. The method of embodiment 36, wherein the immune checkpoint inhibitor is an anti-PD-1 antibody, optionally wherein the immune checkpoint inhibitor is selected from the group consisting of nivolumab (Opdivo), Pembrolizumab (Keytruda), and Cemiplimab (Libtayo).

38. The method of embodiment 35, wherein the immune checkpoint inhibitor inhibits cytotoxic T-lymphocyte-associated antigen 4 (CTLA-4).

39. The method of embodiment 38, wherein the immune checkpoint inhibitor is an anti-CTLA-4 antibody, optionally wherein the immune checkpoint inhibitor is Ipilimumab (Yervoy) or Tremelimumab (Imjudo).

40. The method of embodiment 35, wherein the immune checkpoint inhibitor inhibits programmed cell death protein 1 ligand (PD-L1).

41. The method of embodiment 40, wherein immune checkpoint inhibitor is an anti-PD-L1 antibody, optionally wherein the immune checkpoint inhibitor is selected from the group consisting of Atezolizumab (Tecentriq), avelumab (Bevencio), durvalumab (Imfinzi).

42. The method of any one of embodiments 1-41, wherein the binding agents facilitate the genotype determination of the one or more biomarker genes.

43. The method of embodiment 42, wherein the binding agents comprise reagents capable of determining the genotype by detecting a nucleic acid encoding the biomarker gene or fragments thereof.

44. The method of embodiment 43, wherein the binding agents comprise sequencing reagents.

45. The method of embodiment 44, wherein the sequencing reagents comprise a probe or primer for sequencing the biomarker gene or portion thereof.

46. The method of any one of embodiments 1-41, wherein the binding agents comprise a reagent capable of determining the genotype by detecting a polypeptide.

47. The method of embodiment 46, wherein the binding agents comprise an antibody or an antigen-binding fragment thereof.

48. The method of embodiment 42, wherein the binding agents comprise a label.

49. The method of any one of embodiments 1-48, further comprising administering to the subject a therapy comprising a SHP2 inhibitor or an alternative therapy based on the genotype.

50. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating ARID1A, BAP1, CREBBP, DICER1, KDM6A, KEAP1, KRASG12D, PALB2, or SMARCA4 mutation, (ii) a decreased copy number of ARID1A, BAP1, CREBBP, DICER1, KDM6A, KEAP1, KRASG12D, PALB2, or SMARCA4, or (iii) a decreased expression of ARID1A, BAP1, CREBBP, DICER1, KDM6A, KEAP1, KRASG12D, PALB2, or SMARCA4 mRNA or protein.

51. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mutation, (ii) a decreased copy number of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4, or (iii) a decreased expression of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mRNA or protein.

52. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating BAP1, CUL3, DICER1, KDM6A, KRAS, NCOA6, NF2, PTEN, SMARCA4, TP53, TSC1, or TSC2 mutation, (ii) a decreased copy number of BAP1, CUL3, DICER1, KDM6A, KRAS, NCOA6, NF2, PTEN, SMARCA4, TP53, TSC1,or TSC2, or (iii) a decreased expression of BAP1, CUL3, DICER1, KDM6A, KRAS, NCOA6, NF2, PTEN, SMARCA4, TP53, TSC1,or TSC2 mRNA or protein.

53. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating PBRM1 mutation, (ii) a decreased copy number of PBRM1, or (iii) a decreased expression of PBRM1 mRNA or protein.

54. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating CREBBP, PBRM1, or SMG1 mutation, (ii) a decreased copy number of CREBBP, PBRM1, or SMG1, or (iii) a decreased expression of CREBBP, PBRM1, or SMG1 mRNA or protein.

55. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating EP300, FBXW7, KMT2D, NF1, RB1, RNF43, SETD2, or SMAD4 mutation, (ii) a decreased copy number of EP300, FBXW7, KMT2D, NF1, RB1, RNF43, SETD2, or SMAD4, or (iii) decreased expression of EP300, FBXW7, KMT2D, NF1, RB1, RNF43, SETD2, or SMAD4 mRNA or protein.

56. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mutation, (ii) a decreased copy number of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2, or (iii) decreased expression of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mRNA or protein.

57. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating EP300, FBXW7, or RB1 mutation, (ii) a decreased copy number of EP300, FBXW7, or RB1, or (iii) decreased expression of EP300, FBXW7, or RB1 mRNA or protein

58. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating KEAP1 mutation, (ii) a decreased copy number of EP300, FBXW7, or RB1, or (iii) decreased expression of KEAP1 mRNA or protein.

59. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating ARID1A, BAP1, CREBBP, DICER1, KDM6A, KEAP1, KRASG12D, PALB2, or SMARCA4, (ii) a decreased copy number of ARID1A, BAP1, CREBBP, DICER1, KDM6A, KEAP1, KRASG12D, PALB2, or SMARCA4, (iii) decreased expression of ARID1A, BAP1, CREBBP, DICER1, KDM6A, KEAP1, KRASG12D, PALB2, or SMARCA4 mRNA or protein; (iv) an inactivating EP300, FBXW7, KMT2D, NF1, RB1, RNF43, SETD2, or SMAD4 mutation, (v) a decreased copy number of EP300, FBXW7, KMT2D, NF1, RB1, RNF43, SETD2, or SMAD4, or (vi) a decreased expression of EP300, FBXW7, KMT2D, NF1, RB1, RNF43, SETD2, or SMAD4 mRNA or protein.

60. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mutation, (ii) a decreased copy number of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4, or (iii) a decreased expression of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mRNA or protein; (iv) an inactivating ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mutation, (v) a decreased copy number of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2, or (vi) decreased expression of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mRNA or protein.

61. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating BAP1, CUL3, DICER1, KDM6A, KRAS, NCOA6, NF2, PTEN, SMARCA4, TP53, TSC1, or TSC2 mutation, (ii) a decreased copy number of BAP1, CUL3, DICER1, KDM6A, KRAS, NCOA6, NF2, PTEN, SMARCA4, TP53, TSC1, or TSC2, or (iii) a decreased expression of BAP1, CUL3, DICER1, KDM6A, KRAS, NCOA6, NF2, PTEN, SMARCA4, TP53, TSC1, or TSC2 mRNA or protein; (iv) an inactivating EP300, FBXW7, or RB1 mutation, (v) a decreased copy number of EP300, FBXW7, or RB1, or (vi) decreased expression of EP300, FBXW7, or RB1 mRNA or protein.

62. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating PBRM1 mutation, (ii) a decreased copy number of PBRM1, or (iii) a decreased expression of PBRM1 mRNA or protein; (iv) an inactivating KEAP1 mutation, (v) a decreased copy number of KEAP1, or (vi) decreased expression of KEAP1 mRNA or protein.

63. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating CREBBP, PBRM1, or SMG1 mutation, (ii) a decreased copy number of CREBBP, PBRM1, or SMG1, or (iii) a decreased expression of CREBBP, PBRM1, or SMG1 mRNA or protein; (iv) an inactivating KEAP1 mutation, (v) a decreased copy number of KEAP1, or (vi) decreased expression of KEAP1 mRNA or protein.

64. The method of any one of embodiments 50-63, wherein the cancer is a KRAS-mutant cancer.

65. The method of any one of embodiments 50-64, wherein said tumor sample has previously been determined to comprise a mutation in at least one gene.

66. The method of embodiment 65, wherein the at least one gene is an oncogene.

67. The method of embodiment 66, wherein the oncogene is KRAS.

68. The method of embodiment 65, wherein the at least one gene is a tumor suppressor gene.

69. The method of embodiment 68, wherein the tumor suppressor gene TP53.

70. The method of embodiment 57, further comprising detecting the absence of (i) an inactivating BAP1, CUL3,DICER1, KDM6A, KRAS, NCOA6, NF2, PTEN, SMARCA4, TP53, TSC1, or TSC2 mutation, (ii) a decreased copy number of BAP1, CUL3, DICER1, KDM6A, KRAS, NCOA6, NF2, PTEN, SMARCA4, TP53, TSC1, or TSC2, or (iii) a decreased expression of BAP1, CUL3, DICER1, KDM6A, KRAS, NCOA6, NF2, PTEN, SMARCA4, TP53, TSC1, or TSC2 mRNA or protein.

71. The method of any one of embodiments 53, 54, 58, 62, and 63, wherein said tumor sample has previously been determined to comprise an inactivating TP53 mutation.

72. The method of any one of embodiment 50-71, further comprising obtaining a tumor sample from the subject.

73. The method of any one of embodiments 50-72, wherein the genotype comprises a mutation in the one or more biomarker genes.

74. The method of embodiment 73, wherein the mutation inactivates the biomarker gene.

75. The method of any one of embodiments 59-74, wherein the subject is classified as sensitive to a SHP2 inhibitor treatment.

76. The method of any one of embodiments 59-74, wherein the subject is classified as resistant to a SHP2 inhibitor treatment.

77. The method of any one of embodiments 50-76, further comprising selecting a therapy for the subject.

78. A method of enriching a prospective patient population for subjects likely to respond to a SHP2 inhibitor therapy comprising performing the method of any one of 50 to 63 on two or more individual subjects within the prospective patient population.

79. The method of any one of embodiments 50-78, wherein the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma, and cholangiocarcinoma.

80. The method of embodiment 79, wherein the cancer is colorectal cancer.

81. The method of embodiment 79, wherein the cancer is lung cancer.

82. The method of embodiment 81, wherein the lung cancer is non-small cell lung cancer (NSCLC).

83. The method of embodiment 82, wherein the NSCLC is lung adenocarcinoma.

84. The method of any one of embodiments 50-83, further comprising administering a SHP2 inhibitor therapy to said subject.

85. The method of any one of embodiments 50-84, wherein the SHP2 inhibitor comprises a small molecule.

86. The method of embodiment 85, wherein the SHP2 inhibitor is selected from the group consisting of ERAS-601, BBP-398, RMC-4630, RMC-4550, JAB-3068, JAB-3312, RLY-1971, PF-07284892, TNO-155, ET0038, HBI-2376, HS-10381, and SH3809.

87. The method of any one of embodiments 50-86, wherein the therapy further comprises administering to the subject a second therapy.

88. The method of embodiment 87, wherein the second therapy comprises an agent that affects KRAS activity between inactive guanosine diphosphate (GDP)-bound and active guanosine triphosphate (GTP)-bound states.

89. The method of embodiment 87, wherein the second therapy comprises an agent that targets the immune system directly.

90. The method of embodiment 89, wherein the therapy comprises a programmed cell death protein 1 (PD-1) inhibitor or a colony stimulating factor 1 receptor (CSF-1R) inhibitor.

91. The method of embodiment 89, wherein the second therapy comprises an immune checkpoint inhibitor.

92. The method of embodiment 91, wherein the immune checkpoint inhibitor inhibits programmed cell death protein 1 (PD-1).

93. The method of embodiment 92, wherein the immune checkpoint inhibitor is an anti-PD-1 antibody, optionally wherein the immune checkpoint inhibitor is selected from the group consisting of nivolumab (Opdivo), Pembrolizumab (Keytruda), and Cemiplimab (Libtayo).

94. The method of embodiment 91, wherein the immune checkpoint inhibitor inhibits cytotoxic T-lymphocyte-associated antigen 4 (CTLA-4).

95. The method of embodiment 94, wherein the immune checkpoint inhibitor is an anti-CTLA-4 antibody, optionally wherein the immune checkpoint inhibitor is Ipilimumab (Yervoy) or Tremelimumab (Imjudo).

96. The method of embodiment 91, wherein the immune checkpoint inhibitor inhibits programmed cell death protein 1 ligand (PD-L1).

97. The method of embodiment 96, wherein immune checkpoint inhibitor is an anti-PD-L1 antibody, optionally wherein the immune checkpoint inhibitor is selected from the group consisting of Atezolizumab (Tecentriq), avelumab (Bevencio), durvalumab (Imfinzi).

98. A method of treating cancer in a subject, comprising administering to a subject a therapy comprising a SHP2 inhibitor or an alternative therapy, wherein a tumor sample of the subject comprises the genotype of any one of 50 to 63.

99. The method of any one of embodiments 50-98, further comprising comparing the genotype with a reference genotype.

100. The method of embodiments 50-99, wherein the genotype is reported as a score.

101. The method of any one of embodiments 50-100, wherein determining the genotype comprises genomic profiling.

102. The method of any one of embodiments 50-100, wherein determining the genotype comprises measuring gene expression.

103. The method of embodiment 102, wherein measuring gene expression comprises detection of ribonucleic acids (RNAs) or polypeptides.

104. The method of any one of embodiments 50-103, wherein said determining the genotype of the one or more biomarker genes in the tumor sample comprises contacting the tumor sample with one or more binding agents.

105. The method of embodiment 104, wherein the one or more binding agents facilitate the genotype determination of the one or more biomarker genes.

106. The method of embodiment 104 or 105, wherein the one or more binding agents determine the genotype by detecting a nucleic acid encoding the biomarker gene or fragments thereof.

107. The method of any one of embodiments 104-106, wherein the one or more binding agents comprise sequencing reagents.

108. The method of embodiment 107, wherein the sequencing reagents comprise a probe or primer for sequencing the one or more biomarker genes or a portion thereof.

109. The method of any one of embodiments 104-108, wherein the one or more binding agents comprise a reagent capable of determining the genotype by detecting a polypeptide.

110. The method of embodiment 109, wherein the one or more binding agents comprise an antibody or an antigen-binding fragment thereof.

111. The method of any one of embodiments 104-110, wherein the one or more binding agents comprise a label.

112. A method of treating cancer in a subject, comprising administering to a subject with a SHP2 inhibitor, wherein a tumor sample of the subject comprises (i) an inactivating mutation in one or more of CDKN2A, EP300, KRAS, MGA, EP300, FBXW7, or RB1, (ii) a decreased copy number of one or more of EP300, FBXW7, or RB1, or (iii) decreased expression of mRNA or protein in one or more of EP300, FBXW7, or RB1.

113. The method of embodiment 112, wherein the tumor sample of the subject further comprises absence of (i) an inactivating mutation in one or more of BAP1, CUL3, DICER1, KDM6A, KRAS, NCOA6, NF2, PTEN, SMARCA4, TP53, TSC1, or TSC2, (ii) a decreased copy number of one or more of BAP1, CUL3, DICER1, KDM6A, KRAS, NCOA6, NF2, PTEN, SMARCA4, TP53, TSC1, or TSC2, or (iii) decreased expression of BAP1, CUL3, DICER1, KDM6A, KRAS, NCOA6, NF2, PTEN, SMARCA4, TP53, TSC1, or TSC2 mRNA or protein.

114. A method of treating cancer in a subject, comprising administering to a subject with a SHP2 inhibitor, wherein a tumor sample of the subject comprises (i) an inactivating KEAP1 mutation, (ii) a decreased copy number of KEAP1, or (iii) decreased expression of KEAP1 mRNA or protein.

115. The method of embodiment 114, wherein the tumor sample of the subject further comprises absence of (i) an inactivating mutation in one or more of CREBBP, PBRM1, or SMG1, (ii) a decreased copy number of one or more of CREBBP, PBRM1, or SMG1, or (iii) a decreased expression of CREBBP, PBRM1, or SMG1 mRNA or protein.

116. The method of embodiment 114, wherein the tumor sample of the subject further comprises absence of (i) an inactivating mutation in PBRM1, (ii) a decreased copy number of PBRM1, or (iii) a decreased expression of PBRM1 mRNA or protein.

117. The method of any one of embodiments 112-116, wherein said tumor sample has previously been determined to comprise a mutation in at least one gene.

118. The method of embodiment 117, wherein the at least one gene is an oncogene.

119. The method of embodiment 118, wherein the oncogene is KRAS.

120. The method of embodiment 117, wherein the at least one gene is a tumor suppressor gene

121. The method of embodiment 120, wherein the tumor suppressor gene is TP53.

122. The method of any one of embodiments 114-121, wherein said tumor sample has previously been determined to comprise an inactivating TP53 mutation.

123. The method of any one of embodiments 112-122, wherein the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma and cholangiocarcinoma.

124. The method of embodiment 123, wherein the cancer is colorectal cancer.

125. The method of embodiment 123, wherein the cancer is lung cancer.

126. The method of embodiment 125, wherein the lung cancer is non-small cell lung cancer (NSCLC).

127. The method of embodiment 126, wherein the NSCLC is lung adenocarcinoma.

128. A method of determining effectiveness of a SHP2 inhibitor in reducing tumor size comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; (e) comparing sizes of the first and second inert tumors after the therapy; (f) comparing sizes of the first and second mutant tumors after completion of the therapy, and (g) identifying the mutant tumor genotype as sensitive to the SHP2 inhibitor if the change in tumor size between the first and second inert tumors after the therapy is less than the change in tumor size between the first and second mutant tumors after the therapy.

129. A method of determining effectiveness of a SHP2 inhibitor in reducing tumor size comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; (e) comparing sizes of the first and second inert tumors after the therapy; (f) comparing sizes of the first and second mutant tumors after completion of the therapy, and (g) identifying the mutant tumor genotype as resistant to the SHP2 inhibitor if the change in tumor size between the first and second inert tumors after the therapy is greater than the change in tumor size between the first and second mutant tumors after the therapy.

130. A method of detecting one or more isolated biomarker genes selected from the group consisting of APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

131. A method of detecting one or more isolated biomarker genes selected from the group consisting of ARID1A, BAP1, CREBBP, DICER1, KDM6A, KEAP1, KRASG12D, PALB2, and SMARCA4 in a subject, comprising detecting whether the one or more isolated biomarker genes are present in the tumor sample by contacting a tumor sample of the subject with a binding agent and detecting binding between the one or more isolated biomarker genes and the binding agent, optionally wherein the method further comprises obtaining a tumor sample from the subj ect.

132. A method of detecting one or more isolated biomarker genes selected from the group consisting of BAP1, CUL3, DICER1, KDM6A, KRAS, NCOA6, NF2, PTEN, SMARCA4, TP53, TSC1, and TSC2 in a subject, comprising detecting whether the one or more isolated biomarker genes are present in the tumor sample by contacting a tumor sample of the subject with a binding agent and detecting binding between the one or more isolated biomarker genes and the binding agent, optionally wherein the method further comprises obtaining a tumor sample from the subject.

133. A method of detecting one or more isolated biomarker genes selected from the group consisting of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

134. A method of detecting one or more isolated biomarker genes selected from the group consisting of CREBBP, PBRM1, and SMG1 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

135. A method of detecting PBRM1 in a subject, comprising detecting whether PBRM1 is present in a tumor sample of the subject by contacting the tumor sample with a binding agent and detecting binding between PBRM1 and the binding agent, optionally wherein the method further comprises obtaining a tumor sample from the subject.

136. A method of detecting one or more isolated biomarker genes selected from the group consisting of EP300, FBXW7, KMT2D, NF1, RB1, RNF43, SETD2, and SMAD4 in a subject, comprising detecting whether the one or more isolated biomarker genes are present in a tumor sample of a subject by contacting the tumor sample with a binding agent and detecting binding between the one or more isolated biomarker genes and the binding agent, optionally wherein the method further comprises obtaining a tumor sample from the subject.

137. A method of detecting one or more isolated biomarker genes selected from the group consisting of EP300, FBXW7, and RB1 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

138. A method of detecting one or more isolated biomarker genes selected from the group consisting of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

139. A method of detecting one or more isolated biomarker genes in a subject, comprising detecting whether KEAP1 is present in a tumor sample of the subject by contacting the tumor sample with a binding agent and detecting binding between KEAP1 and the binding agent, optionally wherein the method further comprises obtaining a tumor sample from the subj ect.

140. The method of any one of embodiments 130-139, wherein the binding agent facilitates genotype determination of the one or more biomarker genes.

141. The method of any one of embodiments 130-140, wherein the binding agent determines the genotype by detecting a nucleic acid encoding the biomarker gene or fragments thereof.

142. The method of any one of embodiments 130-141, wherein the binding agent comprises sequencing reagents.

143. The method of embodiment 142, wherein the sequencing reagents comprise a probe or primer for sequencing the biomarker gene or portion thereof.

144. The method of any one of embodiments 130-143, wherein the binding agent comprises a reagent capable of determining the genotype by detecting a polypeptide.

145. The method of embodiment 144, wherein the binding agent comprises an antibody or an antigen-binding fragment thereof.

146. The method of any one of embodiments 130-145, further comprising (d) classifying the human subject as sensitive or resistant to a therapy comprising a SH2 containing protein tyrosine phosphatase-2 inhibitor (SHP2i) based on the detected biomarkers.

### EXAMPLES

### Example 1. Biomarkers of Responsiveness to SHP2 Inhibitor Therapies

This example describes the identification of tumor suppressor genes that are biomarkers of response to SHP2 inhibitor therapies through a method that integrates CRISPR/Cas9-based somatic genome engineering and molecular barcoding into established Cre/Lox-based genetically engineered mouse models of oncogenic Kras-driven lung cancer.

### Generation of Barcoded Lenti-sgRNA/Cre Vector Pool

### 1. Design and Generation of sgRNAs

Lentiviral vectors carrying Cre as well as an sgRNA targeting each of the following genes were generated: Apc, Arid1a, Arid2, Asxl1, Atm, Atrx, Bap1, Brcal, Brca2, Cdkn2a, Chd2, Cic, Cmtr2, Crebbp, Cul3, Dicer1, Dlc1, Dusp4, Ep300, Fat1, Fbxw7, Kdm5c, Kdm6a, Keap1, Kmt2c, Kmt2d, Kras, Lrp1b, Mga, Msh2, Mtap, Ncoa6, Nf1, Nf2, Palb2, Pbrm1, Pcna, Pten, Ptpn11, Ptpn13, Ptprd, Ptprs, Rasa 1, Rb1, Rb1cc1, Rbm10, Rnf43, Setd2, Smad2, Smad4, Smarca4, Smg1, Stag2, Stk11, Tet2, Tgfbr2, Tp53, Tsc1, Tsc2, Usp15, and Zfhx3.

Vectors were also generated carrying inert guides: sgRosa26-1, sgRosa26-2, sgRosa26-3, sgNT-1, sgNT-2, and sgNT-3. All possible 20-bp sgRNAs (using an NGG PAM) targeting each tumor suppressor gene of interest were identified and scored for predicted on-target cutting efficiency using an available sgRNA design/scoring algorithm (Doench et al., Nat Biotechnol 34, 184-191 (2016). https://doi.org/10.1038/nbt.3437). For each tumor suppressor gene, a unique sgRNAs predicted to be the most likely to produce null alleles were selected; preference was given to sgRNAs that were previously validated in vivo (Rogers et al., Nat Methods. 2017 Jul;14(7):737-742. doi: 10.1038/nmeth.4297; Rogers et al., Nat Genet. 2018 Apr;50(4):483-486. doi: 10.1038/s41588-018-0083-2; Winters et al. Nat Commun. 2017 Dec 12;8(1):2053. doi: 10.1038/s41467-017-01519-y. PMID: 29233960; PMCID: PMC5727199, sgRNAs with the highest predicted cutting efficiencies, as well as those targeting exons conserved in all known splice isoforms (ENSEMBL), closest to splice acceptor/splice donor sites, positioned earliest in the gene coding region, occurring upstream of annotated functional domains (InterPro; UniProt), and occurring upstream of known human lung adenocarcinoma mutation sites. Lenti-U6-sgRNA/Cre vectors containing each sgRNA were generated as previously described (Rogers et al., Nat Methods. 2017 Jul;14(7):737-742. doi: 10.1038/nmeth.4297). Briefly, Q5 site-directed mutagenesis (NEB E0554S) was used to insert sgRNAs into the parental lentiviral vector containing the U6 promoter as well as PGK-Cre.

### 2. Barcode Diversification of Lenti-sgRNA/Cre

To enable quantification of the number of cancer cells in individual tumors in parallel using high-throughput sequencing, the Lenti-sgRNA/Cre vectors were diversified with a 46bp multi-component barcode cassette that would be unique to each tumor by virtue of stable integration of the lentiviral vector into the initial transduced cell. This 46 bp DNA barcode cassette was comprised of a known 6-nucleotide ID specific to the vector backbone (vectorID), a 10-nucleotide ID specific to each individual sgRNA (sgID), and a 30-nucleotide random barcode containing 20 degenerate bases (random BC).

The 46 bp barcode cassette for each sgRNA was flanked by universal Illumina® TruSeq adapter sequences and synthesized as single stranded DNA oligos. Forward and reverse primers complimentary to the universal TruSeq sequences and containing 5′ tails with restriction enzyme sites (AscI and NotI) were used in a PCR reaction to generate and amplify double stranded barcode cassettes for cloning. Each Lenti-sgRNA-Cre vector and its matching insert barcode PCR product was digested with AscI and NotI.

To generate a large number of uniquely barcoded vectors, 1 µg of linear vector and 50 ng of insert was ligated with T4 DNA ligase in a 100 µ1 ligation reaction. After 5 hours of incubation at room temperature, ligated DNA was precipitated by centrifugation at 14k for 12 min after adding 5 µ1 Glycogen (5 mg/ml) and 280ul 100% Ethanol into the ligation reaction. The DNA pellet was washed with 80% Ethanol and air dried before being resuspended with 10 µ1 water. This 10 µ1 well-dissolved DNA was transformed into 100 µ1 of Sure Electrical Competent Cells using Biorad electroporation system following their manual. Electroporation-transformed cells were immediately recovered by adding into 5 ml pre-warmed SOC media. From this 5ml cells in SOC medium, 10 µ1 were further diluted with LB ampicillin broth and a final dilution of 1:200K was plated on LB ampicillin plate for incubation at 37° C. The remaining cells in SOC medium were mixed gently and thoroughly before being inoculated into 100 ml LB/Ampicillin broth, shaking at 220 rpm at 37° C. overnight. The next day, colony number on LB/Ampicillin plate were counted to estimate the complexity of each library while 100 ml bacteria culture were pelleted for plasmid purification.

Eight colonies from each library were picked and PCR screened for verification of the specific sgRNA sequence and corresponding barcode sequence among these eight colonies. The final purified library plasmid for each library is again sequence verified.

### Production, Purification, and Titering of Lentivirus

24 hours prior to transfection, 2.4 × 10⁷ 293T cells were plated on 15 cm tissue culture plate. 30 µg of pPack (packaging plasmid mix) and 15 µg of library plasmid DNA were mixed well in 1.5 ml serum free D-MEM medium before equal volume of serum free D-MEM medium containing 90 µl of LipoD293 was added. The resulted mixture was incubated at room temperature for 10-20 min before adding into 293T cells in the 15 cm plate. At 24 hours post-transfection, replace the medium containing complexes with 30 ml of fresh D-MEM medium supplemented with 10% FBS, DNase I (1 U/ml), MgCl₂ (5 mM), and 20 mM HEPES, pH 7.4. The entire virus-containing medium from each plate was collected and filtered through a Nalgene 0.2 µm PES filter at 48 hours post-transfection. The viruses were further concentrated by centrifugation at 18,500 rpm, 4° C. for 2 hours and the pellet was dissolved in 500 ul PBS buffer. 50ul virus aliquots were stored at -80° C.

To determine the titer for packaged library constructs, 1 × 10⁵ LSL-YFP MEF cells were transduced with 1 µl of viruses in 1 ml culture medium containing 5 µg/ml polybrene. Transduced cells were incubated for 72 hours before being collected for FACS analysis to measure the percentage of GFP cells. Control viruses were used in parallel to normalize the virus titers.

### Pooling of Lenti-sgRNA/Cre Vectors

To generate a pool of barcoded Lenti-sgRNA/Cre vectors to generate multiple tumor genotypes within individual mice, barcoded Lenti-sgRNA/Cre vectors targeting Ape, Arid1a, Arid2, Asxl1, Atm, Atrx, Bap1, Brca1, Brca2, Cdkn2a, Chd2, Cic, Cmtr2, Crebbp, Cul3, Dicer1, Dlc1, Dusp4, Ep300, Fat1, Fbxw7, Kdm5c, Kdm6a, Keap1, Kmt2c, Kmt2d, Kras, Lrp1b, Mga, Msh2, Mtap, Ncoa6, Nf1, Nf2, Palb2, Pbrm1, Pcna, Pten, Ptpn11, Ptpn13, Ptprd, Ptprs, Rasa1, Rb1, Rb1cc1, Rbm10, Rnf43, Setd2, Smad2, Smad4, Smarca4, Smg1, Stag2, Stk11, Tet2, Tgfbr2, Tp53, Tsc1, Tsc2, Usp15, and Zfhx3 and those containing the inert, negative control sgRNAs (sgRosa26-1, sgRosa26-2, sgRosa26-3, sgNT-1, sgNT-2, and sgNT-3) were combined such that the viruses would be at equal ratios in relation to their estimated titers. This pool was then diluted with 1× DPBS to reach a final viral titer of 180,000 FU per 60 µL.

### Mice and Tumor Initiation

KrasLSL-G12D (K) and H11LSL-Cas9 (Cas9) mice have been described (Jackson et al., Genes & Dev. 2001. 15: 3243-3248 (doi:10.1101/gad.943001); Chiou et al., Genes & Dev. 2015. 29: 1576-1585 (doi:10.1101/gad.264861.115)). Lung tumors in KrasLSL-G12D/+;H11LSL-Cas9 (KC) mice were initiated via intratracheal delivery of 180,000 functional units (FU) of a lentivirus pool containing barcoded Lenti-U6-sgRNA/PGK-Cre vectors targeting Apc, Arid1a, Arid2, Asxl1, Atm, Atrx, Bap1, Brca1, Brca2, Cdkn2a, Chd2, Cic, Cmtr2, Crebbp, Cul3, Dicer1, Dlc1, Dusp4, Ep300, Fat1, Fbxw7, Kdm5c, Kdm6a, Keap1, Kmt2c, Kmt2d, Kras, Lrp1b, Mga, Msh2, Mtap, Ncoa6, Nf1, Nf2, Palb2, Pbrm1, Pcna, Pten, Ptpn11, Ptpn13, Ptprd, Ptprs, Rasa1, Rb1, Rb1cc1, Rbm10, Rnf43, Setd2, Smad2, Smad4, Smarca4, Smg1, Stag2, Stk11, Tet2, Tgfbr2, Tp53, Tsc1, Tsc2, Usp15, and Zfhx3 plus 6 negative control sgRNAs (three targeting the Rosa26 gene, which are actively cutting but functionally inert, and 3 non-cutting sgRNAs with no expected genomic target [sgNon-Targeting: sgNT]).

In addition, OMI-0017 also contained an extra KrasG12D cDNA vector, to allow for comparison of drug responses of G12C-driven versus G12D-driven tumors in an internally controlled manner as this vector enables the initiation of KrasG12D-driven tumors that do not express KrasG12C in KrasG12C mice. The additional KrasG12D vector drives KrasG12D tumors without additional tumor suppressor gene inactivation and was added as a control for drugs like KrasG12Ci, where KrasG12C tumor will respond very well and KrasG12D tumors within the same mice are not expected to respond.

### Drug Dosing

### Omi-0017

At 15 weeks post induction, Kras^{G12C};Cas9 mice were treated with the following:

Vehicle (n = 39) delivered PO, with once daily dosing (QD), seven consecutive days a week, for three weeks until takedown at 18 weeks post induction.

SHP2 inhibitor RMC-4550 (n = 20) delivered PO, at 30 mg/kg with once daily dosing (QD), seven consecutive days a week, for three weeks until takedown at 18 weeks post induction.

SHP2 inhibitor RMC-4550 (n = 19) delivered PO, at 10 mg/kg with once daily dosing (QD), seven consecutive days a week, for three weeks until takedown at 18 weeks post induction.

SHP2 inhibitor SHP099 (n = 19) delivered PO, at 100 mg/kg with once daily dosing (QD), seven consecutive days a week, for three weeks until takedown at 18 weeks post induction.

SHP2 inhibitor SHP099 (n = 20) delivered PO, at 30 mg/kg with once daily dosing (QD), seven consecutive days a week, for three weeks until takedown at 18 weeks post induction.

### Sc-0016

At 15 weeks post induction, KrasG12C;Cas9 mice were treated with the following:

Vehicle (n = 40) delivered PO, with once daily dosing (QD), seven consecutive days a week, for three weeks until takedown at 18 weeks post induction.

SHP2 inhibitor RMC-4550 (n = 20) delivered PO, at 30 mg/kg with once daily dosing (QD), seven consecutive days a week, for three weeks until takedown at 18 weeks post induction.

### Sc-0015

At 15 weeks post induction, Kras^{G12C};Trp53 ^{flox/flox};Cas9 mice were treated with the following:

Vehicle (n = 59) delivered PO, with once daily dosing (QD), seven consecutive days a week, for three weeks until takedown at 18 weeks post induction.

SHP2 inhibitor RMC-4550 (n = 30) delivered PO, at 30 mg/kg with once daily dosing (QD), seven consecutive days a week, for three weeks until takedown at 18 weeks post induction.

### Dissection of Mouse Lungs

Bulk lung tissue was extracted from euthanized mice as previously described (Rogers et al., Nat Methods. 2017 Jul;14(7):737-742. doi: 10.1038/nmeth.4297). Lung mass measurements were recorded as a proxy for overall lung tumor burden. Lungs were stored at -80° C. prior to subsequent processing.

All mouse experiments were performed in accordance with Animal Care and Use Committee guidelines.

### Generation of Cell Spike-In Controls

DNA barcode cassettes comprised of known 46 bp sequences were flanked by universal Illumina® TruSeq adapter sequences and synthesized as single stranded DNA oligos. Forward and reverse primers complimentary to the universal TruSeq sequences and containing 5′ tails with restriction enzyme sites (Xba1 and BstB1) were used in a PCR reaction to generate and amplify double stranded barcode cassettes for cloning. A lentivector pRCMERP-CMV-MCS-EF1-TagR-Puro and each of the barcode insert PCR products were digested by Xba1 and BstB1 restriction enzymes.

Each digested barcode insert was cloned into linearized vector by T4 DNA ligase and transformed into OmniMax chemical competent cells (Invitrogen). Colonies from each transformation plate were screened by PCR and sequencing. One positive clone from each barcode containing construct was cultured for plasmid DNA extraction.

Virus was packaged from each of the barcoded pRCMERP constructs in 6-well plates using pPack packaging mix and LipoD293 reagent. Virus containing medium were collected at 48 hours post transfection and filtered with Nalgene 0.2 µm PES filter before being frozen down in aliquots at -80° C. Small aliquot of frozen viruses were thawed and added into HEK293 cells in 12-well plate for measuring titer by FACS analysis 72 hours after transduction.

To generate individual cell line containing each barcode construct, virus containing medium was added to HEK293 cells at MOI 0.1 in 10 cm plates. After overnight incubation, cells were recovered in fresh EMEM complete medium for 48 hours before splitting into a new plate containing 1ug/ml puro in complete EMEM medium for puro selection.

After 3 days of puro selection, barcode-containing HEK293 cells were recovered in fresh EMEM complete medium without puro for another 3 days before being further expanded in 10 cm plates. Each established cell line was quality controlled by PCR amplification of the barcode region from genomic DNA to confirm integration of correct barcode sequences.

After cell expansion, cells from each barcoded HEK293 cell line were collected and diluted in PBS buffer containing 0.1%BSA to the desired concentrations. These cell suspensions were aliquoted and frozen down at -80° C.

### Generation of dsDNA Spike-In Controls

DNA barcode cassettes comprised of 46 bp barcode cassettes and flanked by universal Illumina® TruSeq adapter sequences as well as additional buffer sequences to extend their total length to >400bp were generated either by direct synthesis of the double-stranded DNA fragments (GeneWiz, IDT) or synthesis of single-stranded DNA oligos (GeneWiz, IDT) with overlapping complementary regions that were extended and amplified via PCR to create double-stranded DNA products that were then purified. Aliquots of these stock double-stranded DNA fragments were diluted to the desired copy numbers using DNase-free ultra-pure H2O and stored at -20° C.

### Isolation of Genomic DNA From Mouse Lungs

Whole lungs were removed from freezer and allowed to thaw at room temperature. Spikeins were added to each whole lung samples. Added Qiagen Cell Lysis Buffer and proteinase K from Qiagen Gentra PureGene Tissue kit (Cat # 158689) as described in manufacturer protocol. Whole lungs plus spikeins from each mouse were homogenized in the Cell Lysis buffer and Proteinase K solution using a tissue homogenizer (FastPrep-24 5G, MP Biomedicals Cat # 116005500). Homogenized tissue was incubated at 55° C. overnight. To remove RNA from tissues samples, RNase A were added with additional spikeins to whole homogenized tissue. To maintain accurate representation of all tumors, DNA was extracted and alcohol precipitated from the entire lung lysate using Qiagen Gentra PureGene kit as described in manufacturer protocol. More spikeins were added to the resuspended DNA.

### Preparation of sgID-BC Libraries For Sequencing

Libraries were prepared by amplifying the barcode region from 32 µg of genomic DNA per mouse. The barcode region of the integrated Lenti-sgRNA-BC/Cre vectors was PCR amplified using primer pairs that bound the universal Illumina^{®} TruSeq adapters and contained dual unique multiplexing tags. A single-step PCR amplification of barcode regions was used, which was found to be a highly reproducible and quantitative method to determine the number of cancer cells in each tumor. Eight 100 µ1 PCR reactions per mouse (4 µg DNA per reaction) were performed using Q5 HF HS 2x mastermix ((NEB #M0515) with the following PCR program:

| | | | |
|---|---|---|---|
| Step | Temperature (°C) | Time | Cycles |
| Initial Denaturation | 98° C. | 30 seconds | |
| Denaturation | 98° C. | 10 seconds | 30 |
| Annealing | 63° C. | 10 seconds | |
| Extension | 72° C. | 10 seconds | |
| Final Extension | 72° C. | 5 minutes | |
| Hold | 4° C. | ∞ | |

PCR products were purified using SPRI beads. The concentration of purified PCR products from individual mice was determined by TapeStation (Agilent Technologies). Sets of 20-60 samples were pooled at equal ratios. Samples were sequenced on an Illumina® NextSeq and (Cellecta).

### Analysis of Sequencing Data

Paired-end sequencing reads were demultiplexed via dual indexes and adapters sequences were trimmed. Paired-end alignments were constructed between mate-paired reads and library-specific databases of the expected oligonucleotide and tumor barcode insert sequences. These alignments were stringently filtered from downstream analysis if they failed to meet any of several quality criteria, including:
Mismatches between the two mate-pairs, which fully overlap one another, at any location.
Mismatches between the mate-paired reads and expected constant regions of the barcode or spikein to which they best align,
Any indels in alignments between mate-paired reads and the barcode or spikein to which they best align.

Following alignment, errors in paired-end reads were corrected via a simple greedy clustering algorithm:
Reads were dereplicated into read sequence/count tuples, (sᵢ, rᵢ)
These tuples were re-ordered from highest to lowest on the basis of their read abundances, {rᵢ}.
This list of tuples were traversed from i = 1...N, taking one of the following actions for each tuple (sᵢ, rᵢ):
   o If sᵢ is not within a Hamming distance of 1 from any sⱼ with j < i, then (sᵢ, rᵢ) initiates a new cluster.
   o If sⱼ is within a Hamming distance of 1 from some sⱼ with j < i, then it joins the cluster of sⱼ.
The resulting clusters are each considered to represent an error-corrected sequence equal to that of the sequence that founded the cluster and read count equal to the sum of the read counts of the dereplicated reads that are members of the cluster.

Following error correction, the read counts of each unique barcode were converted to tumor cell sizes by dividing the number of error-corrected reads of an oligonucleotide that had been spiked into the sample prior to tissue homogenization and lysis at a fixed, known concentration.

From these collections of tumor sizes across paired groups of KRAS inhibitor-treated and vehicle-treated mice, the relative tumor number (RTN) metric was computed as previously described (Li, C., Lin, W.-Y et al. biorxiv 2020.01.28.923912; doi: https://doi.org/10.1101/2020.01.28.923912).

Namely, shrinkage of inert tumors was estimated by finding the S that matches the median number of tumors in larger than a cutoff L in such paired groups after the vehicle-treated tumor sizes are multiplied by S (S < 1 when KRASi works to shrink tumors). Subsequently, for each non-inert tumor genotype, the ratio of the number of tumors with this genotype larger than L in the control mice to the number of tumors larger than L*S in the treated mice was computed. The resulting ratio was divided by the same ratio computed for the inert tumors, and the log2(·) of this ratio of ratios was determined. This metric, RTN_{score} is expected to be > 0 for resistant genotypes and < 0 for sensitive genotypes.

In order to generate confidence intervals for RTN_{score}, bootstrap re-samplings by (1) sampling mice with replacement from the control and therapy arms to match the original group sizes, and (2) sampling tumors (of all sizes) with replacement from each mouse were generated. For each mouse/tumor bootstrap, the RTN_{score} was re-computed. A genotype was then considered sensitive if the 95th %ile of these bootstrap RTN_{score} values fell below 0, or resistant if the 5th %ile exceeded 0. This bootstrapping procedure was performed at tumor size cutoffs ranging from L=300 cells up to L=10,000 cells.

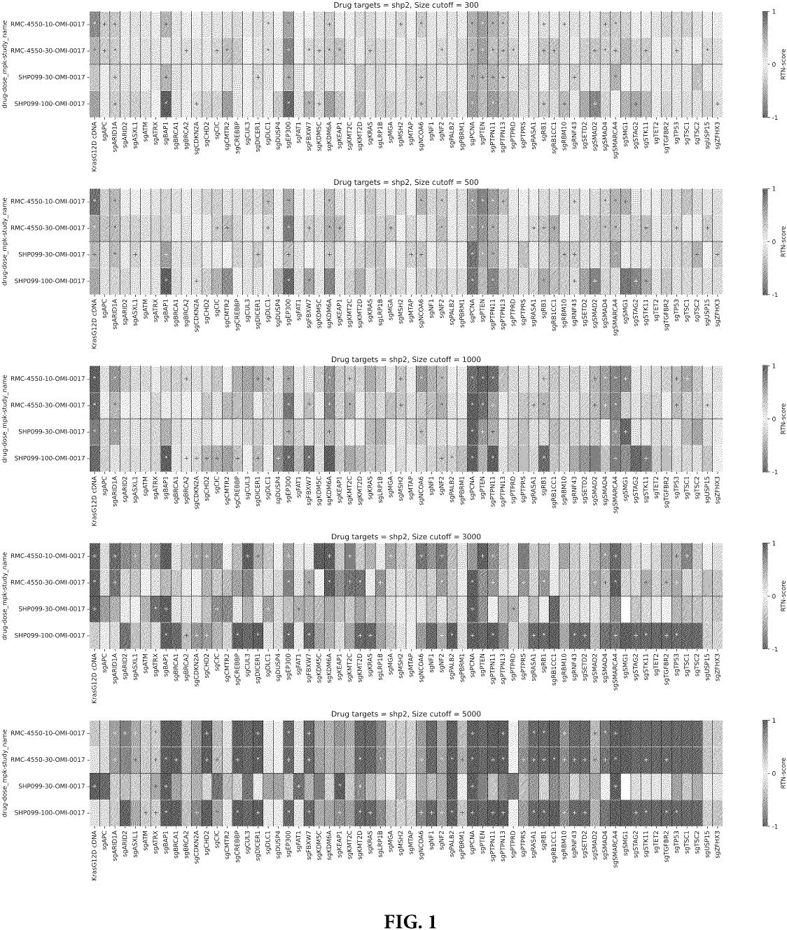

FIG. 1 shows a biomarker heatmap showing the study of pharmacogenomic interactions of SHP2i with inactivation of tumor suppressor genes. Relative tumor number (RTN) > 0 indicates drug resistance, and RTN = 1 corresponds to 2× change in tumor number (larger than each cutoff) relative to change in untreated vs. treated for oncogene-only tumors, while RTN = -1 corresponds to 0.5× change and drug sensitivity . *: p<0.05 and +: p<0.2. Both p-values are two-tailed and based on fraction of bootstraps with RTN scores great or less than 0. Missing cells in heatmap correspond to genotypes that were not assayed in the particular study.

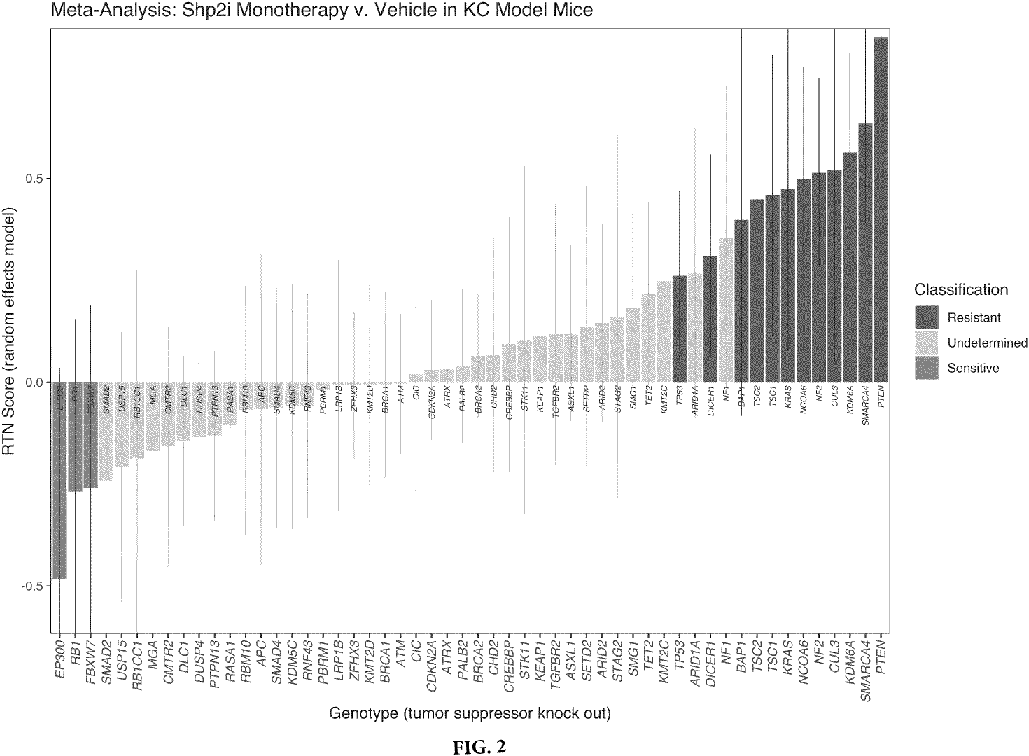

FIG. 2 shows a bar graph depicting aggregated RTN scores and their corresponding about 95% confidence intervals for SHP2 inhibitor therapy. Shading indicates the respective classification memberships, which was defined as follows. Resistant or Sensitive: Family-wise error rate (FWER) less or equal to 0.05 and absolute value of RTN score greater than 0.1. Extended: false discovery rate (FDR) less than or equal to 0.1 and absolute value of RTN score greater than 0.08.

It will be appreciated by those skilled in the art that changes could be made to the embodiments described above without departing from the broad inventive concept thereof. It is understood, therefore, that the present disclosure is not limited to the particular embodiments disclosed, but it is intended to cover modifications within the spirit and scope of the present disclosure as defined by the present description.

What is claimed:

1. A method for determining a genotype of one or more biomarker genes comprising APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a tumor sample of a subject afflicted with cancer, said method comprising (a) contacting the tumor sample with one or more binding agents, each binding agent specific for one of the biomarker genes, and (b) further processing the sample to determine a genotype of the one or more biomarker genes in the tumor sample.

2. The method of claim 1, further comprising (c) classifying the subject as sensitive or resistant to a therapy comprising a SH2 containing protein tyrosine phosphatase-2 inhibitor (SHP2i) based on the genotype of the one or more biomarker genes in the tumor sample of said subject.

3. The method of claim 1 or 2, wherein the biomarker panel comprises ARID1A, BAP1, CREBBP, DICER1, KDM6A, KEAP1, KRASG12D, PALB2, and SMARCA4.

4. The method of claim 3, wherein the biomarker panel further comprises ARID2, ASXL1, ATM, BRCA1, MTAP, NCOA6, NF2, PTEN, PTPN13, and PTPRS.

5. The method of claim 1 or 2, wherein the biomarker panel comprises BAP1, CUL3, DICER1, KDM6A, KRAS, NCOA6, NF2, PTEN, SMARCA4, TP53, TSC1, and TSC2.

6. The method of claim 1 or 2, wherein the biomarker panel comprises EP300, FBXW7, and RB1.

7. The method of claim 6, wherein the biomarker panel further comprises KMT2D, NF1, RNF43, SETD2, and SMAD4.

8. The method of claim 7, wherein the biomarker panel further comprises ATRX, CDKN2A, DLC1, PBRM1, PTPRD, RASA1, RBM10, STK11, TET2, and TGFBR2.

9. The method of claim 1 or 2, wherein the biomarker panel comprises PBRM1.

10. The method of claim 9, wherein the biomarker panel further comprises CREBBP and SMG1.

11. The method of claim 1 or 2, wherein the biomarker panel comprises KEAP1.

12. The method of any one of claims 1 to 11, further comprising obtaining a tumor sample.

13. The method of any one of claims 1 to 12, wherein the genotype comprises a mutation in the one or more biomarker genes.

14. The method of claim 13, wherein the mutation inactivates the biomarker gene.

15. The method of any one of claims 1 to 14, further comprising comparing the genotype with a reference genotype.

16. The method of any one of claims 1 to 15, wherein the genotype is reported as a score.

17. The method of any one of claims 1 to 16, wherein determining the genotype comprises genomic profiling.

18. The method of claim 1, wherein determining the genotype comprises measuring gene expression.

19. The method of claim 18, wherein measuring gene expression comprises detection of ribonucleic acids (RNAs) or polypeptides.

20. The method of any one of claims 2 to 19, wherein the subject is classified as sensitive to SHP2 inhibitor treatment.

21. The method of any one of claims 2 to 19, wherein the subject is classified as resistant to SHP2 inhibitor treatment.

22. The method of any one of claims 1 to 21, wherein the cancer is a KRAS-mutant cancer.

23. The method of any one of claims 1 to 22, wherein the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma and cholangiocarcinoma.

24. The method of claim 23, wherein the cancer is colorectal cancer.

25. The method of claim 23, wherein the cancer is lung cancer.

26. The method of claim 25, wherein the lung cancer is non-small cell lung cancer (NSCLC).

27. The method of claim 26, wherein the NSCLC is lung adenocarcinoma.

28. The method of claim 20, further comprising administering to the subject a SHP2 inhibitor therapy after said genotype determination.

29. The method of any one of claims 2 to 28, wherein the SHP2 inhibitor comprises a small molecule.

30. The method of claim 29, wherein the SHP2 inhibitor is selected from the group consisting of ERAS-601 (Erasca), BBP-398, RMC-4630, RMC-4550, JAB-3068, JAB-3312, RLY-1971, PF-07284892, TNO-155, ET0038, HBI-2376, HS-10381, and SH3809.

31. The method of claim 28, further comprising administering to the subject a second therapy.

32. The method of claim 31, wherein the second therapy comprises an agent that affects KRAS activity between inactive guanosine diphosphate (GDP)-bound and active guanosine triphosphate (GTP)-bound states.

33. The method of claim 32, wherein the second therapy comprises an agent that targets the immune system directly.

34. The method of claim 33, wherein the second therapy comprises a programmed cell death protein 1 (PD-1) inhibitor or a colony stimulating factor 1 receptor (CSF-1R) inhibitor.

35. The method of claim 33, wherein the second therapy comprises an immune checkpoint inhibitor.

36. The method of claim 35, wherein the immune checkpoint inhibitor inhibits programmed cell death protein 1 (PD-1).

37. The method of claim 36, wherein the immune checkpoint inhibitor selected from the group consisting of nivolumab (Opdivo), Pembrolizumab (Keytruda), and Cemiplimab (Libtayo).

38. The method of claim 35, wherein the immune checkpoint inhibitor inhibits cytotoxic T-lymphocyte-associated antigen 4 (CTLA-4).

39. The method of claim 38, wherein the immune checkpoint inhibitor is an anti-CTLA-4 antibody, optionally wherein the immune checkpoint inhibitor is Ipilimumab (Yervoy) or Tremelimumab (Imjudo).

40. The method of claim 35, wherein the immune checkpoint inhibitor inhibits programmed cell death protein 1 ligand (PD-L1).

41. The method of claim 40, wherein immune checkpoint inhibitor is an anti-PD-L1 antibody, optionally wherein the immune checkpoint inhibitor is selected from the group consisting of Atezolizumab (Tecentriq), avelumab (Bevencio), durvalumab (Imfinzi).

42. The method of claim 1, wherein the binding agents can facilitate the genotype determination of the one or more biomarker genes.

43. The method of claim 42, wherein the binding agents comprise reagents capable of determining the genotype by detecting a nucleic acid encoding the biomarker gene or fragments thereof.

44. The method of claim 43, wherein the binding agents comprise sequencing reagents.

45. The method of claim 44, wherein the sequencing reagents comprise a probe or primer for sequencing the biomarker gene or portion thereof.

46. The method of claim 1, wherein the binding agents comprise a reagent capable of determining the genotype by detecting a polypeptide.

47. The method of claim 46, wherein the binding agents comprise an antibody or an antigen-binding fragment thereof.

48. The method of claim 42, wherein the binding agents comprise a label.

49. The method of any one of claims 1 to 48, further comprising administering to the subject a therapy comprising a SHP2 inhibitor or an alternative therapy based on the genotype.

50. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating ARID1A, BAP1, CREBBP, DICER1, KDM6A, KEAP1, KRASG12D, PALB2, or SMARCA4 mutation, (ii) a decreased copy number of ARID1A, BAP1, CREBBP, DICER1, KDM6A, KEAP1, KRASG12D, PALB2, or SMARCA4, or (iii) a decreased expression of ARID1A, BAP1, CREBBP, DICER1, KDM6A, KEAP1, KRASG12D, PALB2, or SMARCA4 mRNA or protein.

51. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mutation, (ii) a decreased copy number of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4, or (iii) a decreased expression of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mRNA or protein.

52. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating BAP1, CUL3, DICER1, KDM6A, KRAS, NCOA6, NF2, PTEN, SMARCA4, TP53, TSC1, or TSC2 mutation, (ii) a decreased copy number of BAP1, CUL3, DICER1, KDM6A, KRAS, NCOA6, NF2, PTEN, SMARCA4, TP53, TSC1, or TSC2, or (iii) a decreased expression of BAP1, CUL3, DICER1, KDM6A, KRAS, NCOA6, NF2, PTEN, SMARCA4, TP53, TSC1, or TSC2 mRNA or protein.

53. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating PBRM1 mutation, (ii) a decreased copy number of PBRM1, or (iii) a decreased expression of PBRM1 mRNA or protein.

54. A method of predicting resistance of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting resistance of tumor cell growth to inhibition by the therapy comprising a SHP2 inhibitor, if the tumor sample comprises (i) an inactivating CREBBP, PBRM1, or SMG1 mutation, (ii) a decreased copy number of CREBBP, PBRM1, or SMG1, or (iii) a decreased expression of CREBBP, PBRM1, or SMG1 mRNA or protein.

55. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating EP300, FBXW7, KMT2D, NF1, RB1, RNF43, SETD2, or SMAD4 mutation, (ii) a decreased copy number of EP300, FBXW7, KMT2D, NF1, RB1, RNF43, SETD2, or SMAD4, or (iii) decreased expression of EP300, FBXW7, KMT2D, NF1, RB1, RNF43, SETD2, or SMAD4 mRNA or protein.

56. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mutation, (ii) a decreased copy number of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2, or (iii) decreased expression of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mRNA or protein.

57. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating EP300, FBXW7, or RB1 mutation, (ii) a decreased copy number of EP300, FBXW7, or RB1, or (iii) decreased expression of EP300, FBXW7, or RB1 mRNA or protein.

58. A method of predicting sensitivity of tumor growth to inhibition by a therapy comprising a SHP2 inhibitor, comprising: (a) detecting in a tumor sample of a subject afflicted with cancer a genotype of one or more biomarker genes; (b) analyzing the genotype of the one or more biomarker genes in the tumor sample; and (c) predicting sensitivity of tumor cell growth to inhibition by the therapy comprising a SHP2 inhibitor, if the tumor sample comprises (i) an inactivating KEAP1 mutation, (ii) a decreased copy number of EP300, FBXW7, or RB1, or (iii) decreased expression of KEAP1 mRNA or protein.

59. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating ARID1A, BAP1, CREBBP, DICER1, KDM6A, KEAP1, KRASG12D, PALB2, or SMARCA4, (ii) a decreased copy number of ARID1A, BAP1, CREBBP, DICER1, KDM6A, KEAP1, KRASG12D, PALB2, or SMARCA4, (iii) decreased expression of ARID1A, BAP1, CREBBP, DICER1, KDM6A, KEAP1, KRASG12D, PALB2, or SMARCA4 mRNA or protein; (iv) an inactivating EP300, FBXW7, KMT2D, NF1, RB1, RNF43, SETD2, or SMAD4 mutation, (v) a decreased copy number of EP300, FBXW7, KMT2D, NF1, RB1, RNF43, SETD2, or SMAD4, or (vi) a decreased expression of EP300, FBXW7, KMT2D, NF1, RB1, RNF43, SETD2, or SMAD4 mRNA or protein.

60. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mutation, (ii) a decreased copy number of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4, or (iii) a decreased expression of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, or SMARCA4 mRNA or protein; (iv) an inactivating ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mutation, (v) a decreased copy number of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2, or (vi) decreased expression of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 mRNA or protein.

61. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating BAP1, CUL3, DICER1, KDM6A, KRAS, NCOA6, NF2, PTEN, SMARCA4, TP53, TSC1, or TSC2 mutation, (ii) a decreased copy number of BAP1, CUL3, DICER1, KDM6A, KRAS, NCOA6, NF2, PTEN, SMARCA4, TP53, TSC1, or TSC2, or (iii) a decreased expression of BAP1, CUL3, DICER1, KDM6A, KRAS, NCOA6, NF2, PTEN, SMARCA4, TP53, TSC1, or TSC2 mRNA or protein; (iv) an inactivating EP300, FBXW7, or RB1 mutation, (v) a decreased copy number of EP300, FBXW7, or RB1, or (vi) decreased expression of EP300, FBXW7, or RB1 mRNA or protein.

62. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating PBRM1 mutation, (ii) a decreased copy number of PBRM1, or (iii) a decreased expression of PBRM1 mRNA or protein; (iv) an inactivating KEAP1 mutation, (v) a decreased copy number of KEAP1, or (vi) decreased expression of KEAP1 mRNA or protein.

63. A method of predicting response of tumor growth to inhibition by a therapy comprising a SHP2 inhibitor, comprising: (a) determining in a tumor sample of a subject afflicted with cancer a genotype of one or more biomarker genes; (b) analyzing the genotype of the one or more biomarker genes in the tumor sample; and (c) predicting the response of tumor cell growth to inhibition by the therapy comprising a SHP2 inhibitor, if the tumor sample comprises (i) an inactivating CREBBP, PBRM1, or SMG1 mutation, (ii) a decreased copy number of PBRM1, or SMG1, or (iii) a decreased expression of CREBBP, PBRM1, or SMG1 mRNA or protein; (iv) an inactivating KEAP1 mutation, (v) a decreased copy number of KEAP1, or (vi) decreased expression of KEAP1 mRNA or protein.

64. The method of any one of claims 50 to 63, wherein the cancer is a KRAS-mutant cancer.

65. The method of any one of claims 50 to 63, wherein said tumor sample has previously been determined to comprise a mutation in at least one gene.

66. The method of claim 65, wherein the at least one gene is an oncogene.

67. The method of claim 66, wherein the oncogene is KRAS.

68. The method of claim 65, wherein the at least one gene is a tumor suppressor gene.

69. The method of claim 68, wherein the tumor suppressor gene TP53.

70. The method of claim 57, further comprising detecting the absence of (i) an inactivating BAP1, CUL3, DICER1, KDM6A, KRAS, NCOA6, NF2, PTEN, SMARCA4, TP53, TSC1, or TSC2 mutation, (ii) a decreased copy number of BAP1, CUL3, DICER1, KDM6A, KRAS, NCOA6, NF2, PTEN, SMARCA4, TP53, TSC1, or TSC2, or (iii) a decreased expression of BAP1, CUL3, DICER1, KDM6A, KRAS, NCOA6, NF2, PTEN, SMARCA4, TP53, TSC1, or TSC2 mRNA or protein.

71. The method of any one of claims 53, 54, 58, 62, and 63, wherein said tumor sample has previously been determined to comprise an inactivating TP53 mutation.

72. The method of any one of claims 50 to 71, further comprising obtaining a tumor sample.

73. The method of any one of claims 50 to 72, wherein the genotype comprises a mutation in the one or more biomarker genes.

74. The method of claim 73, wherein the mutation inactivates the biomarker gene.

75. The method of any one of claims 59 to 74, wherein the subject is classified as sensitive to SHP2 inhibitor treatment.

76. The method of any one of claims 59 to 74, wherein the subject is classified as resistant to SHP2 inhibitor treatment.

77. The method of any one of claims 50 to 76, further comprising selecting a therapy for the subject.

78. A method of enriching a prospective patient population for subjects likely to respond to a SHP2 inhibitor therapy comprising performing the method of any one of claims 50 to 63 on two or more individual subjects within the prospective patient population.

79. The method of any one of claims 50 to 63, wherein the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma and cholangiocarcinoma.

80. The method of claim 79, wherein the cancer is colorectal cancer.

81. The method of claim 79, wherein the cancer is lung cancer.

82. The method of claim 81, wherein the lung cancer is non-small cell lung cancer (NSCLC).

83. The method of claim 82, wherein the NSCLC is lung adenocarcinoma.

84. The method of any one of claims 50 to 83, further comprising administering a SHP2 inhibitor therapy to said subject.

85. The method of any one of claims 50 to 84, wherein the SHP2 inhibitor comprises a small molecule.

86. The method of claim 85, wherein the SHP2 inhibitor is selected from the group ERAS-601, BBP-398, RMC-4630, RMC-4550, JAB-3068, JAB-3312, RLY-1971, PF-07284892, TNO-155, ET0038, HBI-2376, HS-10381, and SH3809.

87. The method of any one of claims 55 to 58, wherein the therapy further comprises administering to the subject a second therapy.

88. The method of claim 87, wherein the second therapy comprises an agent that affects KRAS activity between inactive guanosine diphosphate (GDP)-bound and active guanosine triphosphate (GTP)-bound states.

89. The method of claim 87, wherein the second therapy comprises an agent that targets the immune system directly.

90. The method of claim 89, wherein the therapy comprises a programmed cell death protein 1 (PD-1) inhibitor or a colony stimulating factor 1 receptor (CSF-1R) inhibitor.

91. The method of claim 89, wherein the second therapy comprises an immune checkpoint inhibitor.

92. The method of claim 91, wherein the immune checkpoint inhibitor inhibits programmed cell death protein 1 (PD-1).

93. The method of claim 92, wherein the immune checkpoint inhibitor is an anti-PD-1 antibody, optionally wherein the immune checkpoint inhibitor is selected from the group consisting of nivolumab (Opdivo), Pembrolizumab (Keytruda), and Cemiplimab (Libtayo).

94. The method of claim 91, wherein the immune checkpoint inhibitor inhibits cytotoxic T-lymphocyte-associated antigen 4 (CTLA-4).

95. The method of claim 94, wherein the immune checkpoint inhibitor is an anti-CTLA-4 antibody, optionally wherein the immune checkpoint inhibitor is Ipilimumab (Yervoy) or Tremelimumab (Imjudo).

96. The method of claim 91, wherein the immune checkpoint inhibitor inhibits programmed cell death protein 1 ligand (PD-L1).

97. The method of claim 96, wherein immune checkpoint inhibitor is an anti-PD-L1 antibody, optionally wherein the immune checkpoint inhibitor is selected from the group consisting of Atezolizumab (Tecentriq), avelumab (Bevencio), durvalumab (Imfinzi).

98. A method of treating cancer, comprising administering to a subject a therapy comprising a SHP2 inhibitor or an alternative therapy, wherein a tumor sample of the subject comprises the genotype of any one of claims 50 to 63.

99. The method of any one of claims 50 to 63, further comprising comparing the genotype with a reference genotype.

100. The method of 50 to 99, wherein the genotype is reported as a score.

101. The method of any one of claims 50 to 100, wherein determining the genotype comprises genomic profiling.

102. The method of any one of claims 50 to 100, wherein determining the genotype comprises measuring gene expression.

103. The method of claim 102, wherein measuring gene expression comprises detection of ribonucleic acids (RNAs) or polypeptides.

104. The method of any one of claims 50 to 103, wherein said determining the genotype of the one or more biomarker genes in the tumor sample comprises contacting the tumor sample with one or more binding agents.

105. The method of claim 104, wherein the one or more binding agents can facilitate the genotype determination of the one or more biomarker genes.

106. The method of claim 104 or 105, wherein the one or more binding agents can determine the genotype by detecting a nucleic acid encoding the biomarker gene or fragments thereof.

107. The method of claim 104, wherein the one or more binding agents comprise sequencing reagents.

108. The method of claim 107, wherein the sequencing reagents comprise a probe or primer for sequencing the one or more biomarker genes or a portion thereof.

109. The method of any one of claims 104 to 108, wherein the one or more binding agents comprise a reagent capable of determining the genotype by detecting a polypeptide.

110. The method of claim 109, wherein the one or more binding agents comprise an antibody or an antigen-binding fragment thereof.

111. The method of any one of claims 104 to 110, wherein the one or more binding agents comprise a label.

112. A method of treating cancer in a subject, comprising administering to a subject with a SHP2 inhibitor, wherein a tumor sample of the subject comprises (i) an inactivating mutation in one or more of CDKN2A, EP300, KRAS, MGA, EP300, FBXW7, or RB1, (ii) a decreased copy number of one or more of EP300, FBXW7, or RB1, or (iii) decreased expression of mRNA or protein in one or more of EP300, FBXW7, or RB1.

113. The method of claim 112, wherein the tumor sample of the subject further comprises absence of (i) an inactivating mutation in one or more of BAP1, CUL3, DICER1, KDM6A, KRAS, NCOA6, NF2, PTEN, SMARCA4, TP53, TSC1, or TSC2, (ii) a decreased copy number of one or more of BAP1, CUL3, DICER1, KDM6A, KRAS, NCOA6, NF2, PTEN, SMARCA4, TP53, TSC1, or TSC2, or (iii) decreased expression of BAP1, CUL3, DICER1, KDM6A, KRAS, NCOA6, NF2, PTEN, SMARCA4, TP53, TSC1, or TSC2 mRNA or protein.

114. A method of treating cancer in a subject, comprising administering to a subject with a SHP2 inhibitor, wherein a tumor sample of the subject comprises (i) an inactivating KEAP1 mutation, (ii) a decreased copy number of KEAP1, or (iii) decreased expression of KEAP1 mRNA or protein.

115. The method of claim 114, wherein the tumor sample of the subject further comprises absence of (i) an inactivating mutation in one or more of CREBBP, PBRM1, or SMG1, (ii) a decreased copy number of one or more of CREBBP, PBRM1, or SMG1, or (iii) a decreased expression of CREBBP, PBRM1, or SMG1 mRNA or protein.

116. The method of claim 114, wherein the tumor sample of the subject further comprises absence of (i) an inactivating mutation in PBRM1, (ii) a decreased copy number of PBRM1, or (iii) a decreased expression of PBRM1 mRNA or protein.

117. The method of any one of claims 112 to 116, wherein said tumor sample has previously been determined to comprise a mutation in at least one gene.

118. The method of claim 117, wherein the at least one gene is an oncogene.

119. The method of claim 118, wherein the oncogene is KRAS.

120. The method of claim 117, wherein the at least one gene is a tumor suppressor gene.

121. The method of claim 120, wherein the tumor suppressor gene is TP53.

122. The method of any one of claims 114 to 116, wherein said tumor sample has previously been determined to comprise an inactivating TP53 mutation.

123. The method of any one of claims 112 to 116, wherein the cancer is selected from the group consisting of breast cancer, colorectal cancer, pancreatic adenocarcinoma, lung cancer, uterine corpus endometrial carcinoma, plasma cell myeloma, small intestine adenocarcinoma, gallbladder carcinoma, esophageal squamous cell carcinoma, head and neck squamous cell carcinoma and cholangiocarcinoma.

124. The method of claim 123, wherein the cancer is colorectal cancer.

125. The method of claim 123, wherein the cancer is lung cancer.

126. The method of claim 125, wherein the lung cancer is non-small cell lung cancer (NSCLC).

127. The method of claim 126, wherein the NSCLC is lung adenocarcinoma.

128. A method of determining effectiveness of a SHP2 inhibitor in reducing tumor size comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; (e) comparing sizes of the first and second inert tumors after the therapy; (f) comparing sizes of the first and second mutant tumors after completion of the therapy, and (g) identifying the mutant tumor genotype as sensitive to the SHP2 inhibitor if the change in tumor size between the first and second inert tumors after the therapy is less than the change in tumor size between the first and second mutant tumors after the therapy.

129. A method of determining effectiveness of a SHP2 inhibitor in reducing tumor size comprising: (a) treating a first inert tumor with a control therapy; (b) treating a second inert tumor with a SHP2 inhibitor, wherein the first and second inert tumors comprise identical genotypes; (c) treating a first mutant tumor with the control therapy; (d) treating a second mutant tumor with the SHP2 inhibitor, wherein the first and second mutant tumors comprise identical genotypes; (e) comparing sizes of the first and second inert tumors after the therapy; (f) comparing sizes of the first and second mutant tumors after completion of the therapy, and (g) identifying the mutant tumor genotype as resistant to the SHP2 inhibitor if the change in tumor size between the first and second inert tumors after the therapy is greater than the change in tumor size between the first and second mutant tumors after the therapy.

130. A method of detecting one or more isolated biomarker genes selected from the group consisting of APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

131. A method of detecting one or more isolated biomarker genes selected from the group consisting of ARID1A, BAP1, CREBBP, DICER1, KDM6A, KEAP1, KRASG12D, PALB2, and SMARCA4 in a subject, comprising detecting whether the one or more isolated biomarker genes are present in the tumor sample by contacting a tumor sample of the subject with a binding agent and detecting binding between the one or more isolated biomarker genes and the binding agent, optionally wherein the method further comprises obtaining a tumor sample from the subj ect.

132. A method of detecting one or more isolated biomarker genes selected from the group consisting of BAP1, CUL3, DICER1, KDM6A, KRAS, NCOA6, NF2, PTEN, SMARCA4, TP53, TSC1, and TSC2 in a subject, comprising detecting whether the one or more isolated biomarker genes are present in the tumor sample by contacting a tumor sample of the subject with a binding agent and detecting binding between the one or more isolated biomarker genes and the binding agent, optionally wherein the method further comprises obtaining a tumor sample from the subject.

133. A method of detecting one or more isolated biomarker genes selected from the group consisting of ARID1A, ARID2, ASXL1, ATM, BAP1, BRCA1, CREBBP, DICER1, KDM6A, KEAP1, KRASG12D, MTAP, NCOA6, NF2, PALB2, PTEN, PTPN13, PTPRS, and SMARCA4 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

134. A method of detecting one or more isolated biomarker genes selected from the group consisting of CREBBP, PBRM1, and and SMG1 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

135. A method of detecting PBRM1 in a subject, comprising detecting whether PBRM1 is present in a tumor sample of the subject by contacting the tumor sample with a binding agent and detecting binding between PBRM1 and the binding agent, optionally wherein the method further comprises obtaining a tumor sample from the subject.

136. A method of detecting one or more isolated biomarker genes selected from the group consisting of EP300, FBXW7, KMT2D, NF1, RB1, RNF43, SETD2, and SMAD4 in a subject, comprising detecting whether the one or more isolated biomarker genes are present in a tumor sample of a subject by contacting the tumor sample with a binding agent and detecting binding between the one or more isolated biomarker genes and the binding agent, optionally wherein the method further comprises obtaining a tumor sample from the subject.

137. A method of detecting one or more isolated biomarker genes selected from the group consisting of EP300, FBXW7, and RB1 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

138. A method of detecting one or more isolated biomarker genes selected from the group consisting of ATRX, CDKN2A, DLC1, EP300, FBXW7, KMT2D, NF1, PBRM1, PTPRD, RASA1, RB1, RBM10, RNF43, SETD2, SMAD4, STK11, TET2, TGFBR2, TP53, TSC1, or TSC2 in a subject, comprising detecting whether the one or more isolated biomarker genes are present in a tumor sample of the subject by contacting the tumor sample with a binding agent and detecting binding between the one or more isolated biomarker genes and the binding agent, optionally wherein the method further comprises obtaining a tumor sample from the subject.

139. A method of detecting one or more isolated biomarker genes in a subject, comprising detecting whether KEAP1 is present in a tumor sample of the subject by contacting the tumor sample with a binding agent and detecting binding between KEAP1 and the binding agent, optionally wherein the method further comprises obtaining a tumor sample from the subj ect.

140. The method of any one of claims 130 to 139, wherein the binding agent facilitates genotype determination of the one or more biomarker genes.

141. The method of any one of claims 130 to 140, wherein the binding agent determines the genotype by detecting a nucleic acid encoding the biomarker gene or fragments thereof.

142. The method of any one of claims 130 to 141, wherein the binding agent comprises sequencing reagents.

143. The method of claim 142, wherein the sequencing reagents comprise a probe or primer for sequencing the biomarker gene or portion thereof.

144. The method of any one of claims 130 to 143, wherein the binding agent comprises a reagent capable of determining the genotype by detecting a polypeptide.

145. The method of claim 144, wherein the binding agents comprise an antibody or an antigen-binding fragment thereof.

146. The method of any one of claims 130 to 139, further comprising (d) classifying the human subject as sensitive or resistant to a therapy comprising a SH2 containing protein tyrosine phosphatase-2 inhibitor (SHP2i) based on the detected biomarkers.
